(19) United States
(12) Patent Application Publication
(10) Pub. No.: US 2026/0263125 A1
(43) Pub. Date: Sep. 10, 2026

(54) SCREWS AND PLATES FOR BONE FIXATION

(71) Applicant: Pace Surgical, Inc., Malvern, PA (US)

(72) Inventors: Michael Harper, Bethlehem, PA (US); William S. Rhoda, Media, PA (US); Jeffrey F. O’Donnell, JR., West Chester, PA (US); Stephen S. Soondar, Devon, PA (US); James Christopher Krieg, Haverford, PA (US); Gregory Paul Guyton, Phoenix, MD (US); Nicholas M. Romansky, Malvern, PA (US); Lew Schon, Baltimore, MD (US); Samir Mehta, Haddonfield, NJ (US)

(21) Appl. No.: 19/561,257

(22) Filed: Mar. 9, 2026

Related U.S. Application Data
(60) Provisional application No. 63/769,604, filed on Mar. 10, 2025.

(51) Int. Cl. A61B 17/80; A61B 17/86

(52) U.S. Cl. CPC A61B 17/8057; A61B 17/8605

(57) ABSTRACT

A bone screw and bone plate that allow for enhanced securement of a bone at various angles with reduced friction and necessary torque. The bone screw can include a tri-lobe shaped tip and a transitional thread. The bone plate can include lobes in the inner surface of the opening to secure the bone screw at various angles. The bone screw and bone plate can be positioned with respect to bones or portions of a bone to improve stability.

### INCORPORATION BY REFERENCE TO ANY PRIORITY APPLICATIONS

Any and all applications for which a foreign or domestic priority claim is identified in the Application Data Sheet as filed with the present application are hereby incorporated in their entireties by reference under 37 CFR 1.57. In particular, this application claims priority to the U.S. Provisional Application 63/769604, filed Mar. 10, 2025, which is incorporated by reference herein in its entirety as if fully set forth herein.

### BACKGROUND

A wide variety of bone plates and bone screws can be utilized for internal fixation of bones, for example for bone fractures. Bone screws that can include features to reduce friction and lower the torque requirement for faster insertion are needed and are disclosed herein. Bone screws with a transitional thread are disclosed herein. Bone plates that can include features to improve stability of the bone screw at various angles are needed and are disclosed herein. Bone plates with helically arranged lobes in the openings are disclosed herein.

### SUMMARY OF SOME EMBODIMENTS

Examples of bone screws and bone plates are described herein. In some embodiments, the bone screws can have a thinner thread at a proximal portion of the screw than at a distal portion of the screw. The bone screws may also include a tri-lobe tip. Advantageously, the structure of the bone screw can reduce friction during advancement and can lower the torque requirement for faster insertion. The bone plates may include lobes, for example helically arranged lobes in openings. Advantageously, the lobes can engage screws at various angles to maintain stability of the bone screw at various positions.

In some embodiments, the distal thread profile can be configured to enhance primary fixation in bone, while the proximal thread profile can be configured to reduce frictional contact with bone and/or reduce insertional torque. In some embodiments, the distal thread profile and the proximal thread profile can have substantially equal thread height while differing in one or more other parameters, such as leading surface angle, crest width, thread volume per surface area, and/or flank geometry.

In some embodiments, the bone screw can include a transition portion between the distal thread profile and the proximal thread profile. The transition portion can include an undercut, a taper, a step-off, and/or a curved relief formed in a leading face of the thread. In some embodiments, the transition portion can reduce a surface area of contact between the thread and bone as the screw advances, thereby reducing frictional heating, minimizing bone “packing” ahead of the advancing thread, and/or decreasing the torque required for insertion. In some embodiments, the proximal thread profile can include a greater thread angle (e.g., a steeper leading surface relative to a longitudinal axis of the screw) than the distal thread profile, which can further reduce insertional torque while maintaining distal holding strength.

In some embodiments, bone plates are described herein for use with bone screws. A bone plate can include a main body and one or more openings configured to receive a portion of a bone screw. In some embodiments, an opening can have an inner surface that is generally circular at an upper portion and a lower portion, while including a plurality of lobes extending radially inward from the inner surface. In some embodiments, the lobes can be helically arranged around the opening and can be separated by concave surfaces between adjacent lobes. In some embodiments, the lobes can include threads or threadlike engagement features configured to mate with a threaded screw head and/or to engage a non-threaded screw head by interference and/or surface contact.

In some embodiments, the lobes can include multiple thread starts (e.g., two thread starts) to facilitate rapid engagement between the screw head and the plate opening. In some embodiments, the lobes can be positioned beneath a top surface of the plate and above a bottom surface of the plate to maintain a low profile while still providing locking engagement. Advantageously, the lobes and associated threaded features can engage a screw head at a range of insertion angles relative to a central axis of the opening, thereby providing polyaxial fixation. In some embodiments, the lobed opening can maintain stability of the bone screw at various angles by limiting wobble, increasing thread contact at off-axis orientations, and/or allowing controlled deformation of the plate material during locking to enhance retention.

In some embodiments, a bone screw configured to be secured to a bone can include: a shaft having a proximal end and a distal end, wherein at least the distal end of the shaft has a trilobe shaped profile; a thread on the shaft, the thread including: a first thread portion having a first leading surface disposed at a first thread angle with respect to a longitudinal axis of the bone screw; and a second thread portion having a second leading surface disposed at a second thread angle with respect to a longitudinal axis of the bone screw, wherein the second thread angle is greater than the first thread angle, and wherein the second thread portion is proximal to the first thread portion.

In some embodiments, the thread includes a transition portion between the first thread portion and the second thread portion, the transition portion including a taper in the thread. The bone screw may include a cutting flute on the distal end of the shaft. The transition portion may include an undercut on the thread. In some embodiments, the second thread portion has a greater volume per surface area than the first thread portion, and wherein the first thread portion and second thread portion have an equal height. The bone screw may include three sharp points on a distalmost portion of the distal end. In some embodiments, the second thread portion has a smaller crest width than the first thread portion. In some embodiments, the thread has a wavy shape. In some embodiments, the thread includes serrated lips. The bone screw may include a lumen extending longitudinally through a center of the shaft from the proximal end to the distal end. In some embodiments, a system may include: the bone screw; and a bone plate including an opening configured to receive a portion of the bone screw. The opening of the bone plate may include a plurality of lobes extending from an inner surface of the opening.

In some embodiments, a bone screw configured to be secured to a bone can include: a shaft having a proximal end and a distal end, wherein at least the distal end of the shaft has a trilobe shaped profile; a thread on the shaft, the thread including: a first thread portion having a first volume per surface area; and a second thread portion having a second volume per surface area, wherein the second volume per surface area is greater than the first volume per surface area, wherein the second thread portion is proximal to the first thread portion, and wherein the first thread portion and second thread portion have an equal height.

In some embodiments, the thread includes a transition portion between the first thread portion and the second thread portion, the transition portion including a taper in the thread. The bone screw may include a cutting flute on the distal end of the shaft. In some embodiments, the transition portion includes an undercut on the thread. In some embodiments, the second thread portion has a leading surface with a greater thread angle than a leading surface of the first thread portion. The bone screw may include three sharp points on a distalmost portion of the distal end. In some embodiments, the second thread portion has a smaller crest width than the first thread portion. In some embodiments, the thread has a wavy shape.

In some embodiments, a bone plate configured to support a bone can include: a main body; an opening in the main body, wherein at least an upper portion and a lower portion of the opening include a substantially circular shape; a plurality of lobes extending from an inner surface of the opening; and a concave surface between each lobe of the plurality of lobes, wherein the plurality of lobes are threaded.

In some embodiments, the plurality of lobes include two thread starts. In some embodiments, a first thread start of the two thread starts is on a first lobe of the plurality of lobes and a second thread start of the two thread starts is on a second lobe of the plurality of lobes. In some embodiments, the first lobe is opposite the second lobe along a diameter of the opening. In some embodiments, the plurality of lobes includes six lobes. In some embodiments, the plurality of lobes are beneath a top surface of the main body and above a bottom surface of the main body. The bone plate may include a second opening with a second plurality of lobes. In some embodiments, the opening is in a first segment of the main body and the main body includes a second segment having the second opening. In some embodiments, the second segment is disposed at an obtuse angle with respect to the first segment. In some embodiments, the plurality of lobes includes between 1 and 6 lobes. A system may include: the bone plate; and a bone screw configured to be positioned through the opening of the main body of the bone plate. A thread or shaft of the bone screw may be configured to engage the plurality of lobes of the bone plate.

In some embodiments, a bone plate configured to support a bone can include: a main body; an opening in the main body; a plurality of lobes extending from an inner surface of the opening; and a concave surface between each lobe of the plurality of lobes, wherein the plurality of lobes are threaded and the plurality of lobes include two thread starts.

In some embodiments, the plurality of lobes includes between 1 and 6 lobes. In some embodiments, a first thread start of the two thread starts is on a first lobe of the plurality of lobes and a second thread start of the two thread starts is on a second lobe of the plurality of lobes. In some embodiments, the first lobe is opposite the second lobe along a diameter of the opening. In some embodiments, the plurality of lobes includes six lobes. In some embodiments, the plurality of lobes are beneath a top surface of the main body and above a bottom surface of the main body. The bone plate may include a second opening with a second plurality of lobes. In some embodiments, the opening is in a first segment of the main body and the main body includes a second segment having the second opening.

### BRIEF DESCRIPTION OF THE DRAWINGS

FIG. 1A shows a side view of an example of a bone screw with a trilobe shaped tip, a transitional thread, and a threaded screw head.

FIG. 1B shows another side view of the example of the bone screw of FIG. 1A.

FIG. 1C shows a perspective view of the example of the bone screw of FIG. 1A.

FIG. 1D shows a distal view of the example of the bone screw of FIG. 1A.

FIG. 1E shows a proximal view of the example of the bone screw of FIG. 1A.

FIG. 1F shows a perspective view of the tip of the example of the bone screw of FIG. 1A.

FIGS. 1G-1H show a cross-sectional view of the example of the bone screw of FIG. 1A.

FIGS. 1I-1J show side views of the example of the bone screw of FIG. 1A.

FIG. 2A shows a side view of an example of a head of a bone screw lacking threads.

FIG. 2B shows a perspective view of the example of the head of the bone screw lacking threads of FIG. 2A.

FIG. 2C shows a proximal view of the example of the head of the bone screw lacking threads of FIG. 2A.

FIG. 3A shows an example of a tip and transition zone of a bone screw.

FIG. 3B shows a side view of a thread of the example of the bone screw of FIG. 3A.

FIG. 4A shows a distal view of an example of a bone screw with a sharp trilobe cutting tip and a lumen.

FIG. 4B shows a perspective view of the example of the sharp trilobe cutting tip of FIG. 4A.

FIG. 5A shows a distal view of an example of a bone screw with a smooth trilobe cutting tip and a lumen.

FIG. 5B shows a perspective view of the example of the smooth trilobe cutting tip of FIG. 5A.

FIG. 6A shows a side view of an example of a head of a bone screw.

FIG. 6B shows a perspective view of the example of the head of FIG. 6A.

FIG. 6C shows a proximal view of the example of the head of FIG. 6A.

FIG. 7A shows a cross-sectional side view of an example of a bone screw.

FIG. 7B shows a side view of the example of the thread of the bone screw of FIG. 7A.

FIG. 7C shows a side view of the example of the head of the bone screw of FIG. 7A.

FIG. 8 shows a perspective view of an example of a bone screw with a thread having serrated features.

FIG. 9 shows a perspective view of an example of a bone screw with a wavy thread.

FIG. 10A shows a perspective view of an example of a bone plate opening.

FIG. 10B shows a top view of the example of the bone plate opening of FIG. 10A.

FIG. 11A shows a perspective view of an example of a bone plate.

FIG. 11B shows a top view of the example of the bone plate of FIG. 11A.

FIG. 12A shows a perspective view of an example of a bone plate.

FIG. 12B shows a top view of the example of the bone plate of FIG. 12A.

FIG. 12C shows a top view of the example of the bone plate of FIG. 12A.

FIG. 12D shows a side view of the example of the bone plate of FIG. 12A.

FIG. 13A shows a top view of an example of a bone plate.

FIG. 13B a side view of the example of the bone plate of FIG. 13A.

FIGS. 14-27 show top views of examples of bone plates.

FIG. 28A shows an example of a head of a bone screw engaged with a bone plate.

FIG. 28B shows an example of the bone screw engaged with the bone plate to illustrate the cone of angulation allowed by the interaction of FIG. 28A.

FIG. 29A shows an example of a head of a bone screw with diamond-shaped threads.

FIG. 29B shows an example of a bone plate with diamond-shaped threads of FIG. 29A.

FIG. 29C shows an example of the head of the bone screw interacting with the bone plate with diamond-shaped threads of FIG. 29A.

FIG. 30A shows an example of a head of a bone screw with elliptical engagement features.

FIG. 30B shows an example of a bone plate with elliptical engagement features of FIG. 30A.

FIG. 30C shows an example of the head of the bone screw interacting with the bone plate with elliptical engagement features of FIG. 30A.

FIG. 31A shows a top perspective view an example of a bone plate.

FIG. 31B shows a bottom perspective view of the example of the bone plate of FIG. 31A.

FIGS. 31C-31D show side views of the example of the bone plate of FIG. 31A.

FIG. 31E shows a top view of the example of the bone plate of FIG. 31A.

FIG. 31F shows a bottom view of the example of the bone plate of FIG. 31A.

FIG. 31G shows a back view of the example of the bone plate of FIG. 31A.

FIG. 31H shows a front view of the example of the bone plate of FIG. 31A.

FIG. 31I shows the example of the bone plate of FIG. 31A on a calcaneus.

FIG. 31J shows a perspective view of the opening of the bone plate of FIG. 31A.

FIG. 31K shows a top view of the opening of the bone plate of FIG. 31A.

FIG. 31L shows a bottom view of the opening of the bone plate of FIG. 31A.

FIG. 31M shows a side perspective view of the opening of the bone plate of FIG. 31A.

### DETAILED DESCRIPTION

Disclosed herein are example embodiments of bone screws and bone plates for internal fixation of bones. Though not so limited, the systems, methods, and devices described herein can be used for osteosynthesis and arthrodesis for orthopedic surgery. The screws, or anchors, described herein can be used to secure one or more portions of bone in place, for example when the bone has been fractured or cut. In other examples, the screws can be used to provide stability between two bones in order to weld or fuse the bones together. The bone plates can be used to provide rigidity between a plurality of bones or portions of bone. The openings in the bone plates can be used to position the screws at the desired location on the bones or portions of bone. The bone screws and bone plates may be used to fixate bones in an extremity of a patient. In some examples, the bone screws and plates may be used on a lower extremity, for example a femur, tibia, fibula, and/or a bone of the foot. In some examples, the bone screws and plates may be used on an upper extremity, for example a humerus, radius, ulna, clavicle, and/or bone of the hand or wrist. In some examples, the bone screws and plates may be used on a spine, pelvis, and/or hip of a patient.

A bone screw can be inserted into a hole in the bone plate and threaded into the bone to position the bone against the plate. This can lock the bone in a position that allows for healing of a fracture.

### Bone Crews:

Any embodiments of the bone screws described herein can include features to reduce insertional torque. Accordingly, in any embodiments disclosed herein, the bone screw 100 can be configured to reduce the insertional torque, or prevailing torque, required to form a hole in bone or to insert the screw in the bone.

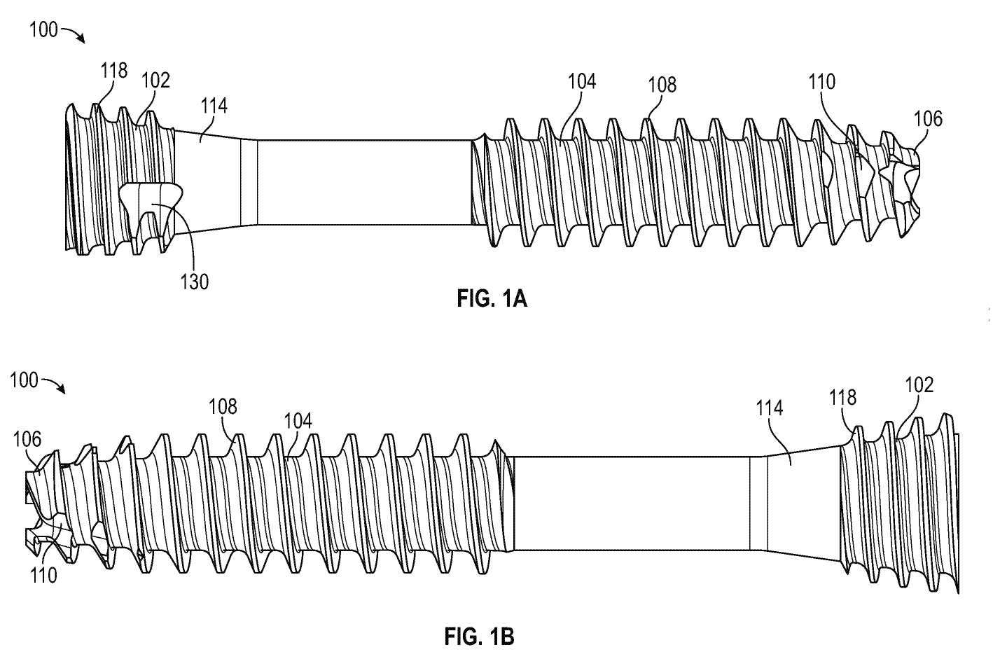

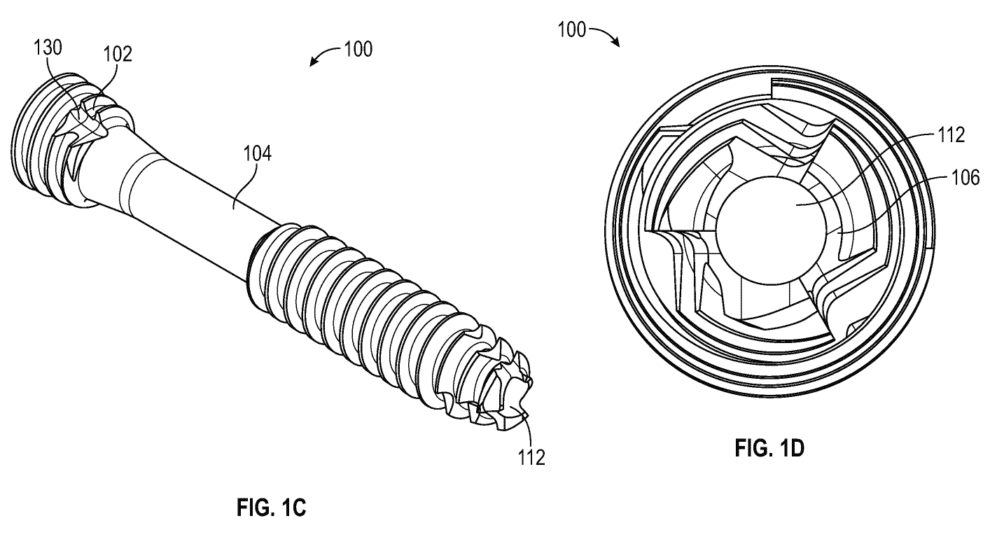

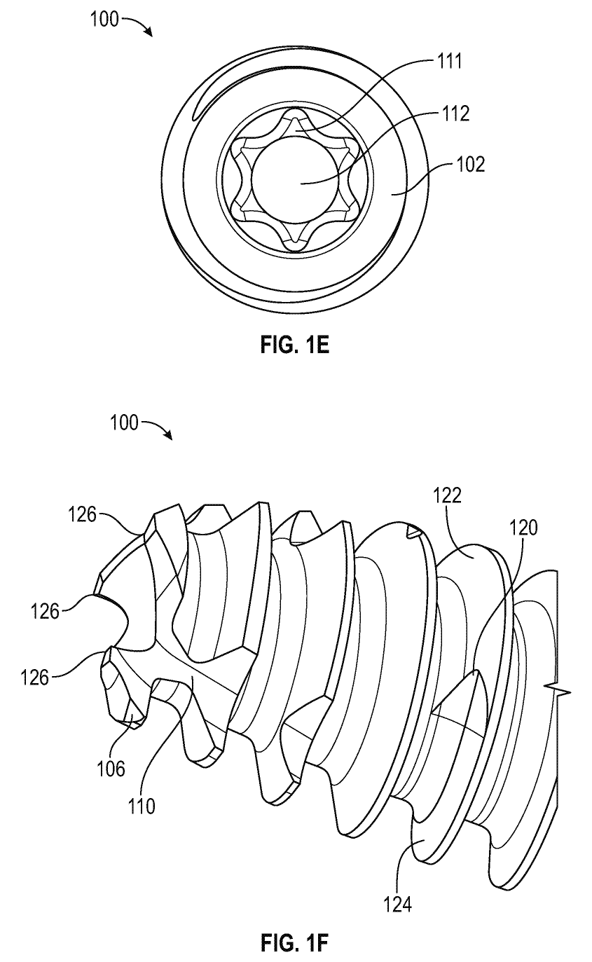

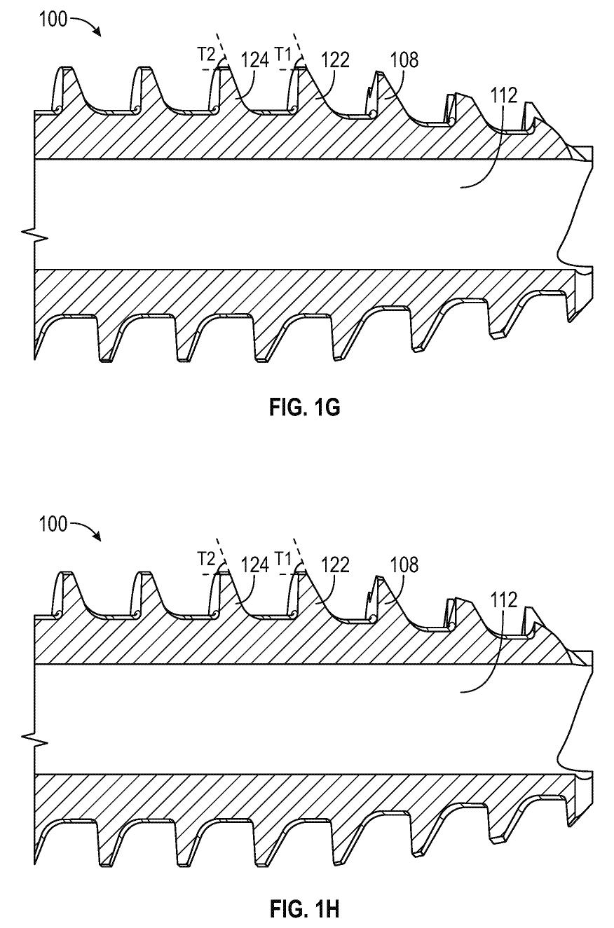

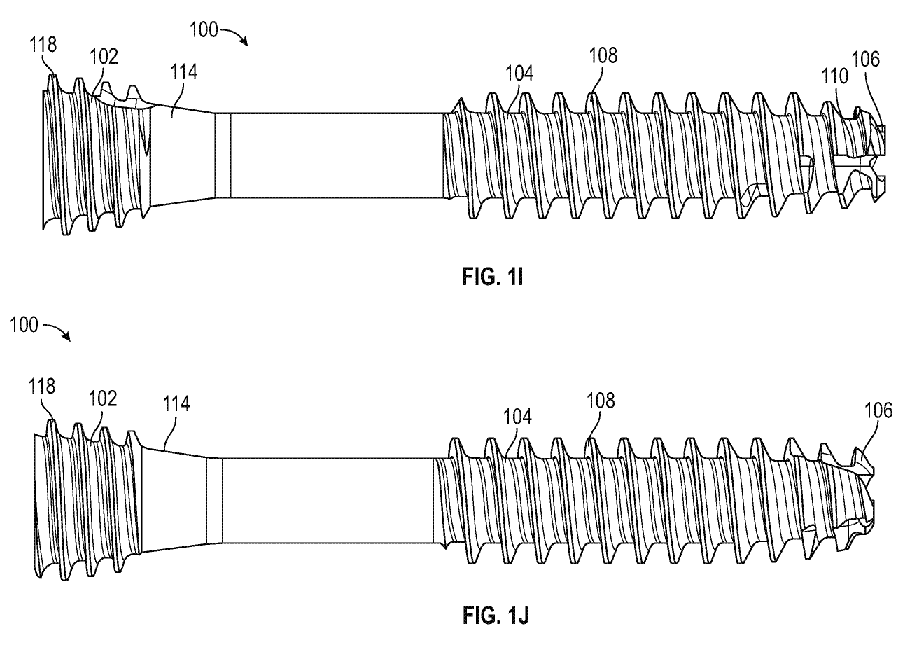

FIG. 1A shows a side view of an example embodiment of a bone screw 100 with a trilobe shaped tip 106, a transitional thread 108, and a threaded screw head 102. FIG. 1B shows another side view of the example of the bone screw 100. FIG. 1C shows a perspective view of the example of the bone screw 100. FIG. 1D shows a distal view of the example of the bone screw 100. FIG. 1E shows a proximal view of the example of the bone screw 100. FIG. 1F shows a perspective view of the tip 106 of the example of the bone screw 100. FIGS. 1G-1H show cross-sectional views of the example of the bone screw 100 of FIG. 1A. FIGS. 1I-1J show side views of the example of the bone screw 100 of FIG. 1A.

In some embodiments, the bone screw 100 can have a screw head 102 at the proximal end, a shaft 104 extending from a distal end of the screw head 102, and a tip 106 at the distal end of the shaft 104. The bone screw 100 can have a thread 108 around the shaft 104.

In some embodiments, the bone screw 100 can have cutting flutes 110 in the thread 108 and/or the shaft 104. In some embodiments, the cutting flutes 110 can be longitudinal cuts along the perimeter of the thread 108 and/or the shaft 104. In some embodiments, the bone screw 100 can have three cutting flutes 110 around the tip 106 and the adjacent threads 108 and/or portion of the shaft 104. In some embodiments, the bone screw 100 can have 1-5 cutting flutes 110 around the tip 106, threads 108, and/or distal portion of the shaft 104. In some embodiments, the bone screw 100 can have 1-10 cutting flutes 110 around the tip 106, threads 108, and/or distal portion of the shaft 104. Advantageously, the cutting flutes 110 can help create threads in the bone without irritating nearby soft tissue. The cutting flutes 110 can also maximize or enhance screw holding power. The thread 108 can have a thread root at the radially inward point and a thread crest at the radially outward point. The thread 108 can follow a helical path.

In some embodiments, the tip 106 of the bone screw 100 can have a trilobe shape. In some embodiments, the tip 106 of the bone screw 100 can have a traditional end portion without the trilobe feature. In some embodiments, the shaft 104 of the bone screw 100 can have a trilobe shape. In some embodiments, a trilobe shape can include three curved sides and three round corners. For example, the cross-section of the tip 106 and/or the shaft 104 can resemble a triangle with three curved sides of approximately equal length with three round corners. Advantageously, the trilobe shape of the tip 106 can reduce the insertional torque, or prevailing torque, required to form a hole in bone. In some embodiments, the trilobe tip 106 can reduce the insertional torque of the bone screw 100 by 10% or approximately 10% compared to a conventional cylindrical screw. In some embodiments, the trilobe tip 106 can reduce the insertional torque of the bone screw 100 by from 5% or approximately 5% to 15% or approximately 15% compared to a conventional cylindrical screw. In some embodiments, the trilobe tip 106 can reduce the insertional torque of the bone screw 100 by from 1% or approximately 1% and 25% or approximately 25% compared to a conventional cylindrical screw. Additionally, in any embodiments disclosed herein, the trilobe tip 106 can provide resistance to vibrational loosening of the bone screw 100.

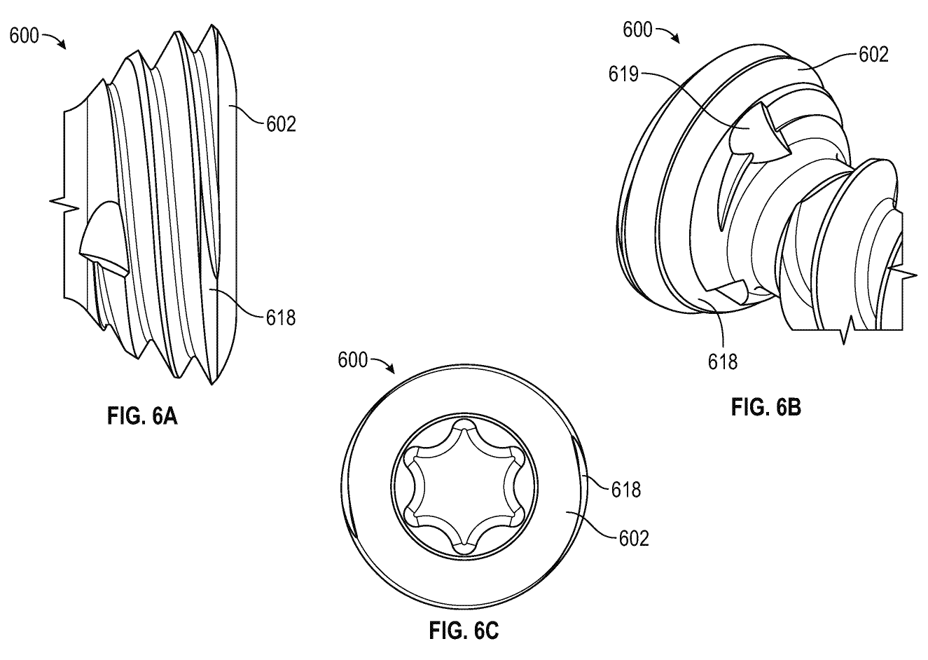

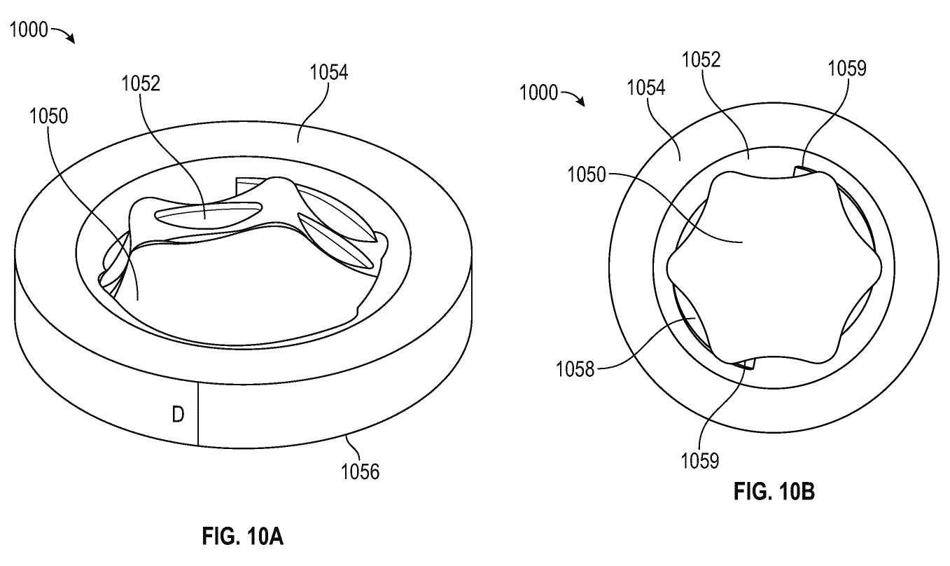

As shown in FIGS. 1A and 1B, a shank 114 of the shaft 104 can taper radially outward from the shaft 104 toward the head 102. In some embodiments, the head 102 can include threads 118 configured to engage a bone. The thread 118 on the head 102 can engage a first bone or first portion of bone, and the thread 108 on the shaft 104 can engage a second bone or second portion of bone. The threads 108,118 can cause compression between the first bone and second bone or first portion of bone and second portion of bone. Advantageously, compressing the bones or portions of bone can secure them together. In some embodiments, the thread 118 can include any of the features of the thread 108. In some embodiments, the bone screw 100 can be a headless compression screw. In other examples, the head 102 can include threads configured to mate with a hole of a bone plate, as described with respect to FIG. 6A-6B. For example and without limitation, the bone screw 100 can include features to secure to a bone plate described with respect to FIGS. 10A-10B, 11A-11B, 12A-12D, 13A-13B, or 14-27. In some embodiments, the threads 118 on the head 102 can include a cutting flute 130.

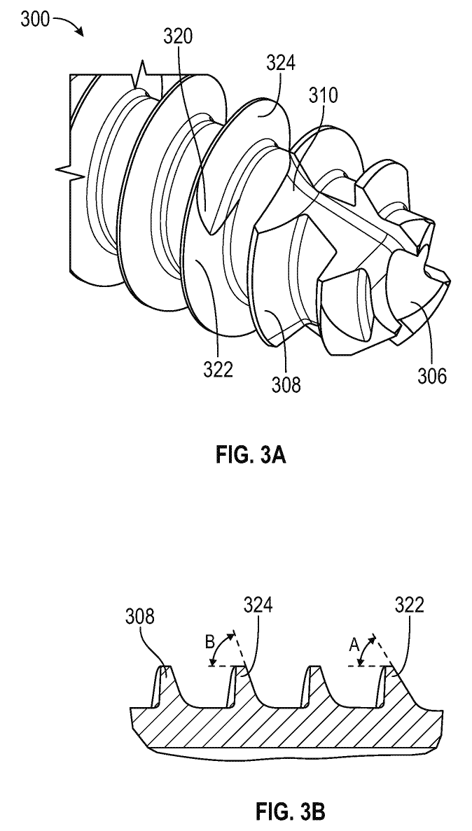

As shown in FIGS. 1C, 1D, and 1E, in some embodiments, the bone screw 100 can be cannulated. In other embodiments, the bone screw 100 can be non-cannulated. The bone screw 100 can have a lumen 112 through the center of the screw along the longitudinal axis. In some embodiments, the bone screw 100 can be hollow to provide stability while allowing for the insertion of a guidewire through the lumen 112. In some embodiments, the bone screw may lack a lumen as shown with respect to FIGS. 3A, 4B, 5B, and 6A-6B.

As shown in FIG. 1E, in some embodiments, the bone screw 100 can include a drive 111, or recess. In some embodiments, the drive 111 can be a wavy shape, for example having lobes. In some embodiments, the drive 111 can have 6 lobes. In some embodiments, the drive 111 can have 2-10 lobes.

As shown in FIG. 1F, in some embodiments, the thread 108 can include a transition zone 120 between a distal thread profile 122 and a proximal thread profile 124. The transition zone 120 can be an undercut, taper, or a step-off in the thread 108. The transition zone 120 can be cut into the leading face of the thread 108. In some embodiments, the transition zone 120 can be a round, hemispherical, or curved cut in the leading face of the thread 108.

As shown in FIG. 1G, in some embodiments, the distal thread profile 122 can have a lower thread angle than the proximal thread profile 124. The distal thread profile 122 can have a first thread angle T1 and the proximal thread profile can have a second thread angle T2. In some examples, at least one of the first thread angle T1 or second thread angle T2 can be the angle of the leading surface with respect to a longitudinal axis of the bone screw 100. In some examples, at least one of the first thread angle T1 or second thread angle T2 can be the lesser or acute angle taken with respect to the longitudinal axis of the bone screw 100. For example, the distal thread profile 122 can have a thread angle T1 of 60° or approximately 60° and the proximal thread profile can have a thread angle T2 of 70° or approximately 70°. In some embodiments, the distal thread profile 122 can have a thread angle T1 of at least 50° and/or less than or equal to 70°. In some embodiments, the distal thread profile 122 can have a thread angle T1 of at least 30° and/or less than or equal to 90°. In some embodiments, the proximal thread profile 124 can have a thread angle T2 of at least 60° and/or less than or equal to 80°. In some embodiments, the proximal thread profile 124 can have a thread angle T2 of at least 40° and/or less than or equal to 90°. In some embodiments, the distal thread profile 122 can have a greater crest width than the proximal thread profile 124. In some embodiments, the distal thread profile 122 and/or the proximal thread profile 124 can have a crest width of greater than or equal to 0.1 mm and/or less than or equal to 0.5 mm. In some embodiments, the distal thread profile 122 and/or the proximal thread profile 124 can have a crest width of greater than or equal to 0 mm and/or less than or equal to 1 mm. In some embodiments, the distal thread profile 122 can have a greater volume per surface area than the proximal thread profile 124. The distal thread profile 122 may allow for increased primary stability or mechanical grip. For example, the distal thread profile 122 can have a greater cross-sectional area than the proximal thread profile 124 when the greater diameter of the distal thread profile 122 and the proximal thread profile 124 are equal. The distal thread profile 122 may have a greater cross-sectional area than the proximal thread profile 124 when the height of the distal thread profile 122 and the proximal thread profile 124 are equal. The height and greater diameter of the distal thread profile 122 and the proximal thread profile 124 may be equal at the position in which the threads are not tapered. In some examples, a thread portion comprises an individual thread.

In some embodiments, the transition between the distal thread profile 122 and the proximal thread profile 124 can reduce friction by approximately 15% compared to a conventional screw. In some embodiments, the transition between the distal thread profile 122 and the proximal thread profile 124 can reduce friction by from 10% or approximately 10% to 20% or approximately 20% compared to a conventional screw. In some embodiments, the transition between the distal thread profile 122 and the proximal thread profile 124 can reduce friction by between 1% or approximately 1% and 30% or approximately 30% compared to a conventional screw.

In some embodiments, the combination of the transition between the distal thread profile 122 and the proximal thread profile 124 and the trilobe shaped tip 106 can reduce friction by 25% or approximately 25%. In some embodiments, the combination of the transition between the distal thread profile 122 and the proximal thread profile 124 and the trilobe shaped tip 106 can reduce friction by from 15% or approximately 15% to 35% or approximately 35%. In some embodiments, the combination of the transition between the distal thread profile 122 and the proximal thread profile 124 and the trilobe shaped tip 106 can reduce friction by from approximately 1% to approximately 60%.

In some embodiments, the transition zone 120 can be cut into the thread 108 with a whirler tool. In other embodiments, the transition zone 120 can be integrally formed or cut with a different tool. In some embodiments, the curved shape of the transition zone 120 can soften the transition between the distal thread profile 122 and the proximal thread profile 124. Advantageously, this can reduce damage to surrounding tissues and improve securement of the bone screw 100 in the bone. In some embodiments, the transition zone 120 can reduce the surface area of contact with the bone such that the distal thread profile 122 has a greater surface area of contact with the bone than the proximal thread profile 124.

As shown in FIG. 1F, the tip 106 can have distalmost projections 126. In some embodiments, the distalmost projections can include two or more surfaces separated by at least one recess, space, gap, or cutting flute. In some embodiments, the distalmost projections 126 can be sharp, and can catch or cut into the bone. Advantageously, the distalmost projections 126 can reduce the force necessary to insert the bone screw 100. The portion of the tip 106 surrounding distalmost projections 126 can scrape the bone as the bone screw 100 enters the bone. In some embodiments, the scraped bone can fill gaps or depressions of the bone adjacent to the thread 108. For example, the scraped bone can fill gaps or depressions formed by the distal thread profile 122 once the proximal thread profile 124 is adjacent to the gaps or depressions. The scraped bone and/or bone material can ride up the proximal thread profile 124, which can have less volume per surface area than the distal thread profile 122. Advantageously, the transition between the distal thread profile 122 and the proximal thread profile can prevent the scraped bone and/or bone material from interfering with the subsequent cuts into the bone. Advantageously, scraped bone and/or bone material can reform into the bone to better secure the bone screw 100 in the bone. In some embodiments, the bone screw 100 can have multiple threads 108, thread starts, or transition zones 120.

In some embodiments, the bone screw 100 can have a length from the proximal end to the distal end of 21 mm or approximately 21 mm. In some embodiments, the bone screw 100 can have a length from the proximal end to the distal end of approximately 15-25 mm. In some embodiments, the bone screw 100 can have a length from the proximal end to the distal end of approximately 10-30 mm. In some embodiments, the bone screw 100 can have a length from the proximal end to the distal end of approximately 1-40 mm. In some embodiments, the bone screw 100 can have a length from the proximal end to the distal end of approximately 1-60 mm. In some embodiments, the bone screw 100 can have a length from the proximal end to the distal end of approximately 1-80 mm.

In some embodiments, a bone screw 100 for use on an upper extremity of a patient may have a length from approximately 8 mm to approximately 50 mm. A bone screw 100 for use on an upper extremity of a patient may have a length from approximately 4 mm to approximately 75 mm. A bone screw 100 for use on an upper extremity of a patient may have a length from approximately 2 mm to approximately 100 mm. In some embodiments, a bone screw 100 for use on an upper extremity of a patient may have a diameter from approximately 2 mm to approximately 4 mm. A bone screw 100 for use on an upper extremity of a patient may have a diameter from approximately 1 mm to approximately 8 mm. A bone screw 100 for use on an upper extremity of a patient may have a diameter from approximately 0.1 mm to approximately 12 mm.

In some embodiments, the thread 108 can be disposed on approximately 60% of the length of the shaft 104. In some embodiments, the thread 108 can be disposed on approximately 50-70% of the length of the shaft 104. In some embodiments, the thread 108 can be disposed on approximately 30-90% of the length of the shaft 104. In some embodiments, the thread 108 can be disposed on approximately 10-100% of the length of the shaft 104.

In some embodiments, the bone screw 100 can have a major diameter, or outer diameter of the thread 108, of 2.5 mm or approximately 2.5 mm. In some embodiments, the bone screw 100 can have a major diameter of at least approximately 2 mm and/or less than or equal to approximately 3 mm. In some embodiments, the bone screw 100 can have a major diameter of at least approximately 1 mm and/or less than or equal to approximately 5 mm. In some embodiments, the bone screw 100 can have a major diameter of at least approximately 0.5 mm and/or less than or equal to approximately 10 mm. In some embodiments, the bone screw 100 can have a minor diameter, or diameter of the shaft 104, of 1.5 mm or approximately 1.5 mm. In some embodiments, the bone screw 100 can have a minor diameter of at least approximately 1 mm and/or less than or equal to approximately 2 mm. In some embodiments, the bone screw 100 can have a minor diameter of at least approximately 0.5 mm and/or less than or equal to approximately 4 mm. In some embodiments, the bone screw 100 can have a major diameter of at least approximately 0.25 mm and/or less than or equal to approximately 8 mm.

In some embodiments, the reduced insertional torque can be achieved by configuring the thread 108 with a first thread geometry in a distal region and a second thread geometry in a proximal region, with a relief or transition therebetween. For example, the distal region can include a comparatively aggressive thread form configured to form or cut bone and provide primary fixation, while the proximal region can include a comparatively non-aggressive thread form configured to reduce sliding contact with bone as the screw advances. In some embodiments, the proximal region can be described as having a reduced thread “mass,” such as a reduced crest width, a reduced flank-to-flank thickness, a reduced projected contact area of the leading surface, and/or a reduced cross-sectional area of the thread at a given major diameter, thereby reducing frictional engagement and the prevailing torque required for insertion.

In some embodiments, the reduced insertional torque can be further achieved by configuring the shaft 104 and/or tip 106 with a non-cylindrical profile that reduces circumferential rubbing and provides clearance for cut bone. For example, the distal end of the shaft 104 and/or the tip 106 can be described as having a trilobe profile, a tri-flat profile with radiused corners, a three-arc profile, or a generally triangular profile with convex sides, such that the screw defines alternating contact regions and clearance regions about the circumference. In some embodiments, the trilobe profile can be combined with one or more longitudinal cutting flutes 110 and/or distalmost projections 126 such that, during insertion, the distal end preferentially cuts and displaces bone while reducing continuous surface-to-surface rubbing. Advantageously, these alternative descriptions capture that the screw geometry can reduce the effective contact area and facilitate chip evacuation, thereby lowering friction and the torque required to advance the screw into bone.

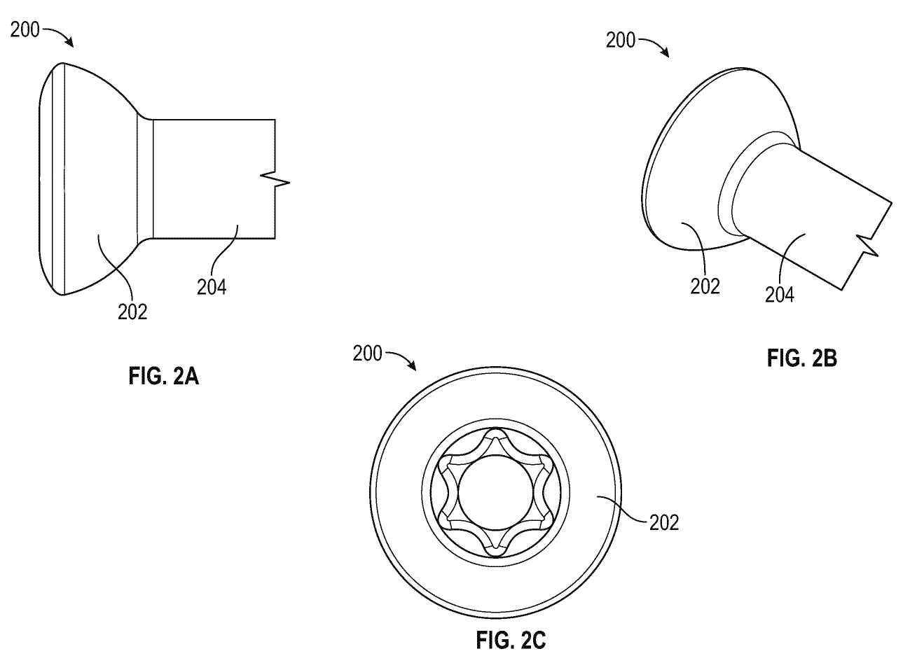

FIG. 2A shows a side view of an example of a head 202 of a bone screw 200. FIG. 2B shows a perspective view of the example of the head 202. FIG. 2C shows a proximal view of the example of the head 202.

The bone screw 200 can include any of the features of the bone screw 100 as described with respect to FIGS. 1A-1J. In some embodiments, the head 202 can lack threads. The shaft 204 can lack a taper distal to the head 202.

In some embodiments, the head 202 of the bone screw 200 can secure to a bone plate without threads. In some embodiments, the bone screw 200 can be positioned in a bone without a bone plate. In some embodiments, the head 202 of the bone screw 200 can be a spherical head that presses against the bone to compress the bone.

FIG. 3A shows an example of a tip 306 and transition zone 320 of a bone screw 300. FIG. 3B shows a side view of a thread 308 of the example of the bone screw 300.

The bone screw 300 can include any of the features of the bone screw 100,200 as described herein.

As shown in FIG. 3A, the bone screw 300 can lack a lumen, thus having a closed tip 306. The tip 306 can have a sharp distal end. In some embodiments, the thread 308 can include a transition zone 320 that where the distal thread profile 322 transitions to the proximal thread profile 324. The bone screw 300 can include cutting flutes 310.

As shown in FIG. 3B, in some embodiments, the thread 308 can transition from a distal thread profile 322 with a thread angle A to a proximal thread profile 324 with a thread angle B. In some embodiments, the distal thread profile 122 can have a lower thread angle than the proximal thread profile 124. For example, the distal thread profile 122 can have a thread angle A of 60° or approximately 60° and the proximal thread profile can have a thread angle B of 70° or approximately 70°. In some embodiments, the distal thread profile 122 can have a thread angle A of at least 50° and/or less than or equal to 70°. In some embodiments, the distal thread profile 122 can have a thread angle A of at least 30° and/or less than or equal to 90°. In some embodiments, the proximal thread profile 124 can have a thread angle B of at least 60° and/or less than or equal to 80°. In some embodiments, the proximal thread profile 124 can have a thread angle B of at least 40° and/or less than or equal to 90°.

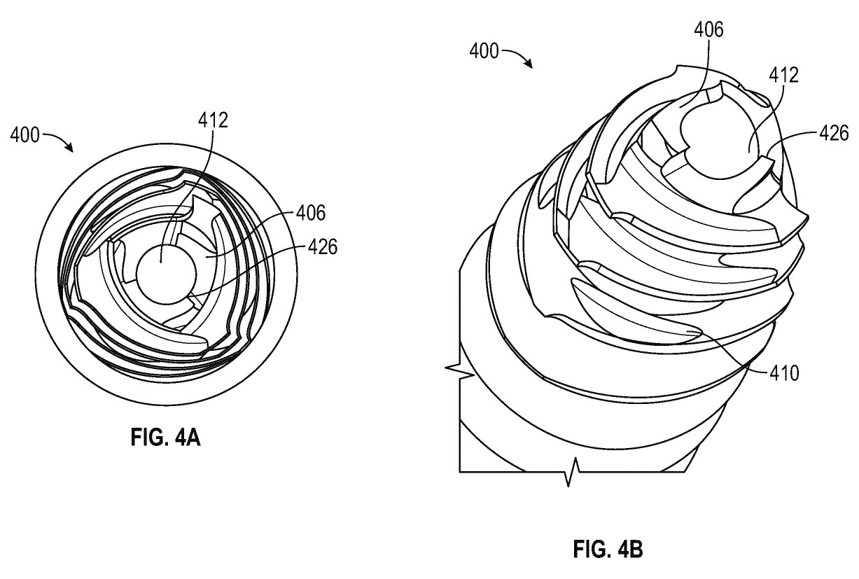

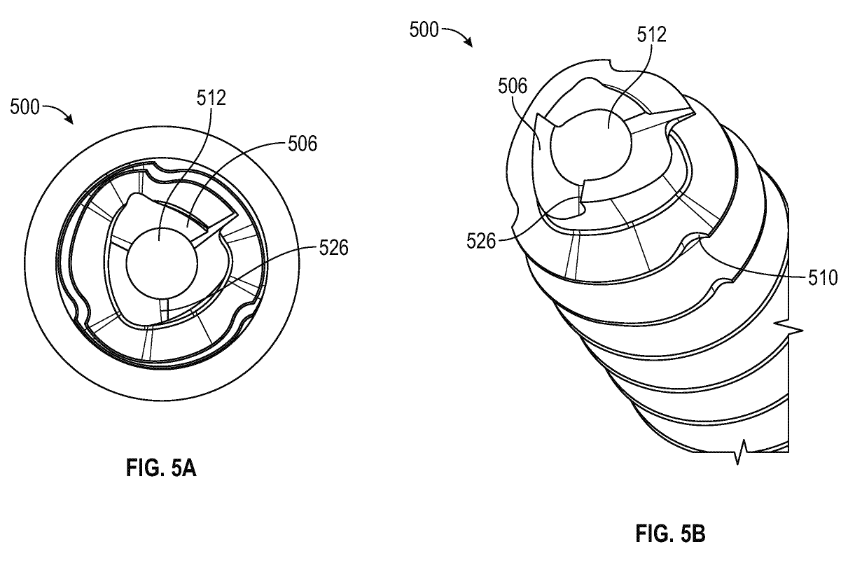

FIG. 4A shows a distal view of an example of a bone screw 400 with a sharp trilobe cutting tip 406 and a lumen 412. FIG. 4B shows a perspective view of the example of the sharp trilobe cutting tip 406.

The bone screw 400 can include any of the features of the bone screw 100,200,300 as described herein.

In some embodiments, the bone screw 400 can have a trilobe shaped tip 406. The trilobe shaped tip 406 can include three sharp distalmost projections 426. For example, each of the distalmost projections 426 can have a sharp slope proximal to the distalmost point such that the tips are optimized for cutting. Advantageously, the sharp distalmost projections 426 and trilobe shaped tip 406 can reduce friction during insertion and the necessary torque. The bone screw 400 can include smaller, or minimized, cutting flutes 410 to ease thread forming.

As shown in FIG. 4A, the bone screw 400 can be cannulated with a lumen 412. The lumen 412 can extend through a central longitudinal axis of the bone screw 400.

FIG. 5A shows a distal view of an example of a bone screw 500 with a smooth trilobe cutting tip 506 and a lumen 512. FIG. 5B shows a perspective view of the example of the smooth trilobe cutting tip 506.

The bone screw 500 can include any of the features of the bone screw 100,200,300,400 as described herein.

In some embodiments, the bone screw 500 can have a trilobe shaped tip 506. The trilobe shaped tip 506 can include three smooth distalmost projections 526. For example, each of the distalmost projections 526 can have a smooth slope proximal to the distalmost point such that the tips are optimized for easy entry into the bone. Advantageously, the smooth distalmost projections 526 and trilobe shaped tip 506 can reduce friction during insertion and the necessary torque. The bone screw 500 can include smaller, or minimized, cutting flutes 510 to ease thread forming.

FIG. 6A shows a side view of an example of a head 602 of a bone screw 600 for mating with a bone plate. FIG. 6B shows a perspective view of the example of the head 602. FIG. 6C shows a proximal view of the example of the head 602.

The bone screw 600 can include any of the features of the bone screw 100,200,300,400,500 as described herein. Embodiments of the screw described herein can include the head 602 of FIGS. 6A-6C with the shaft, thread, tip, and/or cutting flutes as described with respect to FIGS. 1A-1F.

In some embodiments, the threads 618 of the head 602 can be enhanced for coupling with a bone plate having features described with respect to FIGS. 10A-10B, 11A-11B, 12A-12D, 13A-13B, or 14-27. The head 602 can be optimized with engaging lobes of a bone plate. In some embodiments, the head 602 can have a tapered multi-lead thread 618. The multi-lead thread 618 of the head 602 can interact with lobes of a mating plate for locking and/or limiting angular movement. In some embodiments, the multi-lead thread 618 of the head 602 can be tapered. As shown in FIG. 6B, the head 602 can include a relief feature 619. The relief feature 619 can be a curved cut in the thread 618 and/or the head 602. The relief feature 619 can act as a cutting feature for under the plate. The relief feature 619 can aid in engagement between the bone screw 600 and the bone plate.

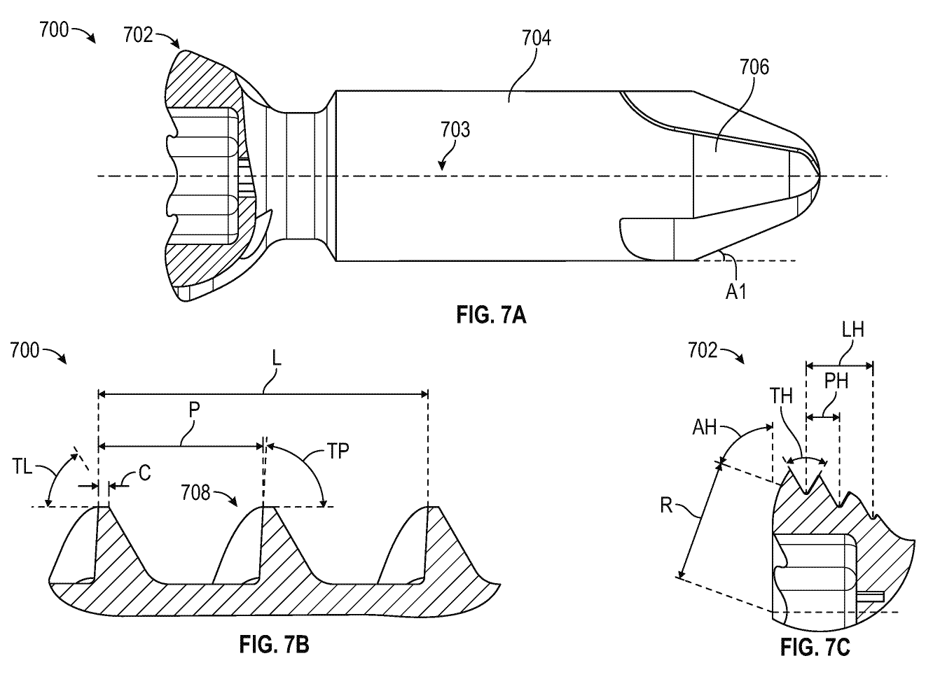

FIG. 7A shows a cross-sectional side view of an example of a bone screw 700 with the threads not shown. FIG. 7B shows a side view of the threads of the example of the bone screw 700. FIG. 7C shows a side view of the example of the head 702 of the bone screw 700.

The bone screw 700 can include any of the features of the bone screw 100, 200, 300, 400, 500, 600 as described herein. In some embodiments, the bone screw 700 can include a head 702 at a proximal end and a tip 706 at a distal end. As shown in FIG. 7A, the bone screw 700 can have a central longitudinal axis 703. The tip 706 of the bone screw 700 can taper from the shaft 704 at an angle A1. In some embodiments, the tip 706 of the bone screw 700 can taper from the shaft 704 at an angle A1 of 45° or approximately 45°. In some embodiments, the tip 706 of the bone screw 700 can taper from the shaft 704 at an angle A1 of from approximately 30° to approximately 60°.

In some embodiments, as shown in FIG. 7B, the thread 708 can have a leading flank angle TL and a proximal flank angle TP. The leading flank angle TL can vary as described with respect to FIGS. 1A-1J. In some embodiments, the leading flank angle TL is 60° or approximately 60°. In some embodiments, the leading flank angle TL is from approximately 40° to approximately 80°. In some embodiments, the proximal flank angle TP is 87.5° or approximately 87.5°. In some embodiments, the proximal flank angle TP is from approximately 70° to approximately 100°. In some embodiments, the thread 708 can have a crest C of approximately 0.1 mm. In some embodiments, the thread 708 can have a crest C of from approximately 0.05 mm to approximately 0.5 mm. In some embodiments, the thread 708 can have a pitch P of 1.5 mm or approximately 1.5 mm. In some embodiments, the thread 708 can have a pitch P of from approximately 1 mm to approximately 2 mm. In some embodiments, the thread 708 can have a pitch P of from approximately 0.5 mm to approximately 4 mm. In some embodiments, the thread 708 can have a lead L of approximately 3 mm. In some embodiments, the thread 708 can have a lead L of from approximately 1 mm to approximately 5 mm.

In some embodiments, as shown in FIG. 7C, the head 702 can be engineered to fit with a bone plate described herein. In some embodiments, the head 702 can be engineered to avoid going too far through the bone plate. In some embodiments, the head 702 can have a radius R of 2.2 mm or approximately 2.2 mm. In some embodiments, the head 702 can have a radius R of from approximately 1 mm to 4 mm. In some embodiments, the head 702 can be tapered at an angle AH of 70° or approximately 70°. In some embodiments, the head 702 can be tapered at an angle AH of from approximately 60° to approximately 80°. In some embodiments, the head 702 can have a pitch PH of 0.6 mm or approximately 0.6 mm. In some embodiments, the head 702 can have a pitch PH of from approximately 0.1 mm to approximately 2 mm. In some embodiments, the head 702 can have a lead LH of 1.2 mm or approximately 1.2 mm. In some embodiments, the head 702 can have a lead LH of from approximately 0.5 mm to approximately 3 mm. In some embodiments, the thread angle TH of the head 702 is 60° or approximately 60°. In some embodiments, the thread angle TH of the head 702 is from approximately 40° to approximately 80°.

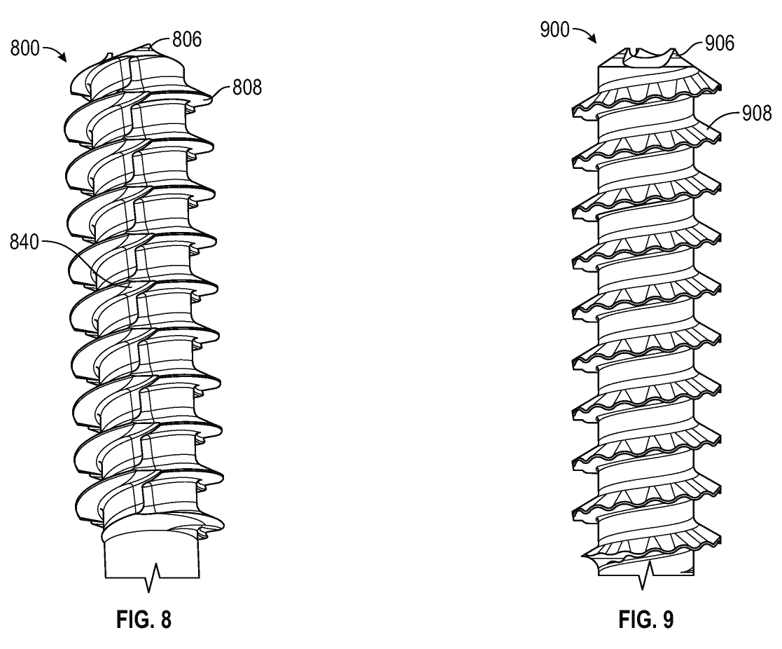

FIG. 8 shows a perspective view of an example of a bone screw 800 with a thread 808 having serrated features 840.

The bone screw 800 can include any of the features of the bone screw 100, 200, 300, 400, 500, 600, 700 as described herein.

In some embodiments, the thread 808 can include serrated features 840, or lips. The thread 808 can include serrated features 840 that are aligned along a longitudinal axis of the bone screw 800. The thread 808 can include a serrated feature 840 at each point of the thread 808 along the longitudinal axis.

In some embodiments, the serrated features 840 can reduce friction during insertion. In some embodiments, the serrated features 840 can be a helical cut in the thread 808. The helical cut in the thread 808 can cause the bone to skip off the edge of the thread 808 and not contact the adjacent thread 808 for part of a rotation. In some embodiments, the serrated feature 840 can be a 2 mm helical cut that tapers back to the helical path of the thread. In some embodiments, the serrated feature 840 can be a 1-5 mm helical cut that tapers back to the helical path of the thread. Reducing friction during insertion can reduce the torque necessary to position the bone screw 800 in bone. The serrated features 840 can be incrementally varied radially to alter and shift the geometry of the bone screw 800 along the length of the screw. Advantageously, this can increase the pull-out force of the bone screw 800. In some embodiments, the serrated features 840 can form part of the hole in the bone to reduce the surface area of contact between the thread 808 and the bone. In some embodiments, there can be a cutting face at the screw tip 806.

FIG. 9 shows a perspective view of an example of a bone screw 900 with a wavy thread 908.

The bone screw 900 can include any of the features of the bone screw 100, 200, 300, 400, 500, 600, 700, 800 as described herein.

In some embodiments, the bone screw 900 can include a wavy thread 908. The wavy thread 908 can be a thread with a wavy structure. For example, the wavy thread 908 can have a curved path of alternating crests (this may refer to wave crests, not thread crests) and troughs. In some embodiments, the entire thread 908 is structured as waves. For example, the thread root of the thread 908 can follow a helical path, the thread crest can follow a path that alternates between a minimum and a maximum distance away from the helical path. In some embodiments, the thread root and the thread crest can follow a path that alternates between a minimum and a maximum distance away from a helical path. In some embodiments, the minimum and maximum distance from the helical path can be from 0.15 mm to 0.5 mm from the helical path. In some embodiments, the minimum and maximum distance from the helical path can be from 0.05 mm to 0.8 mm from the helical path. In some embodiments, the thread 908 can be partially wavy. In some embodiments, the portion of the wavy thread 908 most distal on the leading surface, or the crest of the wave, can form the hole in the bone to reduce the surface area of contact between the more proximal portions of the leading surface of the wavy thread 908 and the bone. In some embodiments, the thread crests, or the most radially outward surface of the thread 908, can have a wavy shape.

In some embodiments, the wavy thread 908 can reduce friction during insertion. Reducing friction during insertion can reduce the torque necessary to position the bone screw 900 in bone. The wavy thread 908 can be incrementally varied radially to alter and shift the geometry of the bone screw 900 along the length of the screw. Advantageously, this can increase the pull-out force of the bone screw 900. In some embodiments, there can be a cutting face at the screw tip 906.

### Bone Plates:

FIG. 10A shows a perspective view of an example of a bone plate 1000 illustrating an opening 1050. FIG. 10B shows a top view of the example of the bone plate opening 1050.

A bone plate 1000 can be used to secure a bone screw in place. The bone plate 1000 can support one or more bones to maintain the bones in a desired position. Advantageously, the bone plate 1000 described herein can provide enhanced strength with a reduced profile due to the lobes 1052 in the opening 1050. The bone plate 1000 can be used to secure a bone screw including any of the features of the bone screw 100, 200, 300, 400, 500, 600, 700, 800, 900 as described herein.

In some embodiments, the bone plate 1000 can include an opening 1050 in the main body of the bone plate. The opening 1050 can be a substantially circular opening in the main body. The bone plate 1000 can include lobes 1052, or flanges, surrounding the opening 1050. In some embodiments, the lobes 1052 can take the form of individual segmented features configured to interact with a bone screw. In some embodiments, the lobes 1052 can have convex profiles with concave profiles or spaces therebetween. The lobes 1052 can be curved surfaces between a top surface 1054 of the bone plate 1000 and a bottom surface 1056 of the bone plate 1000. The lobes 1052 may protrude from an inner surface of the opening 1050 beneath the top surface 1054. The lobes 1052 may protrude from an inner surface of the opening 1050 above the bottom surface 1056. The lobes 1052 can be helically arranged around the opening 1050. In some embodiments, the lobes 1052 can be threaded or have threads or threadlike features 1058 thereon so that the threaded features extend substantially radially inwardly. The thread 1058 can be cut into the profile of the lobes 1052 along a spiral path. In some embodiments, the bone plate 1000 can include 6 lobes, which may be described as hexalobe shape of the opening 1050. In some embodiments, the bone plate 1000 can include from 3 to 9 lobes. In some embodiments, the bone plate 1000 can include from 1 to 12 lobes. In some embodiments, the lobes 1052 can be radially inward curves separated by radially outward curves, such that the lobed opening 1050 is shaped like a continuous wave.

In some embodiments, the lobes 1052 of the opening 1050 can have two thread starts 1059. In some embodiments, the lobes 1052 of the opening 1050 can have 1-5 thread starts 1059. In some embodiments, the thread starts 1059 can be on lobes 1052 opposite one another. Advantageously, having multiple thread starts 1059 in the lobe 1052 can allow the bone screw to be secured easily and effectively. In some embodiments, the radially outward curves can have smaller radii than the radially inward lobes 1052.

In some embodiments, the lobes 1052 can secure the head of a bone screw at a variety of angles. In some embodiments, the lobes 1052 can secure a threaded head of a bone screw. In some embodiments, the lobes 1052 can secure a head of a bone screw lacking threads. In some embodiments, the lobes 1052 can allow a user to secure a bone screw at an angle within a 15° or approximately 15° cone of angulation. In some embodiments, the lobes 1052 can allow a user to secure a bone screw at an angle within a cone of angulation of at least approximately 10° and/or less than or equal to approximately 20°. In some embodiments, the lobes 1052 can allow a user to secure a bone screw at an angle within a cone of angulation of at least approximately 5° and/or less than or equal to approximately 30°. In some embodiments, the lobes 1052 can allow a user to secure a bone screw at an angle within a cone of angulation of at least approximately 1° and/or less than or equal to approximately 60°.

In some embodiments, the bone plate 1000 can have a low profile, or be substantially thin. For example, the bone plate 1000 can have a depth D of 1.1 mm or approximately 1.1 mm. In some embodiments, the bone plate 1000 can have a depth D of from approximately 0.5 mm to approximately 1.5 mm. In some embodiments, the bone plate 1000 can have a depth D of from approximately 0.1 mm to approximately 2 mm. Advantageously, the bone plate 1000 can have a high strength to profile ratio due to the shape of the lobes 1052. Advantageously, this can allow for enhanced stability of the bone screw without requiring a thicker plate, thus limiting the interference with surrounding structures.

In some embodiments, a bone plate 1000 for use on an upper extremity of a patient may have a thickness from approximately 1.1 mm to approximately 1.8 mm. A bone plate 1000 for use on an upper extremity of a patient may have a thickness from approximately .5 mm to approximately 3 mm. A bone plate 1000 for use on an upper extremity of a patient may have a thickness from approximately .1 mm to approximately 5 mm.

In some embodiments, the lobes 1052 can be discrete inwardly projecting lands, pads, or ribs that interrupt an otherwise generally circular inner wall of the opening 1050. Each lobe 1052 can define a locally thickened region of plate material having an arcuate radially inward face configured to bear against a screw head. In some embodiments, the lobes 1052 can be circumferentially spaced about the opening 1050 to define alternating engagement regions (at the lobes 1052) and clearance regions (at the concave surfaces between lobes 1052), such that the screw head can be seated at multiple angular orientations while maintaining multiple points of contact.

In some embodiments, the lobes 1052 can be described as helically indexed projections, wherein each lobe 1052 includes an engagement surface that is axially offset relative to an adjacent lobe 1052 along the thickness direction of the plate. For example, the lobes 1052 can form a partial internal helical ramp or spiral track within the opening 1050, such that rotation of a screw head causes progressive engagement at one or more lobes 1052 and draws the screw head axially toward the plate. This helical arrangement may provide a polyaxial locking effect by permitting the screw head to engage the opening 1050 even when the screw axis is tilted relative to a central axis of the opening 1050.

In some embodiments, the lobes 1052 can include threadlike features 1058 that are not necessarily continuous threads along the entire circumference of the opening 1050, but instead are segmented internal thread portions provided on the lobes 1052. In some embodiments, the threadlike features 1058 can be partial thread turns, interrupted thread segments, scalloped thread segments, or multi-start thread segments disposed on the lobes 1052. In some embodiments, the threadlike features 1058 can include angled ridges, peaks, crests, or flank surfaces configured to mate with a corresponding external thread on a screw head, thereby resisting back-out and limiting wobble when the screw is seated at a non-orthogonal insertion angle.

In some embodiments, the lobes 1052 can be configured to engage a screw head by non-threaded locking mechanisms, such as interference, wedging, biting, and/or controlled deformation. For example, each lobe 1052 can include a chamfered lead-in, a radiused lead-in, or a tapered bearing surface that transitions into a more abrupt retention surface such that the screw head can cam the lobe 1052 outward during insertion and thereafter be retained by elastic recovery and/or plastic deformation of the plate material. In some embodiments, the lobes 1052 can be deformable locking tabs or compliant retention segments configured to conform locally to the surface of the screw head, thereby increasing contact area and stability across a range of insertion angles.

In some embodiments, the geometry of the lobes 1052 and the concave surfaces between lobes 1052 can be configured to define a socket that supports the screw head in a polyaxial manner. For example, the concave surfaces can act as relief pockets to accommodate a tilted screw head and to avoid point-loading at a single circumferential location, while two or more lobes 1052 provide spaced-apart engagement that stabilizes the screw head against rocking. In some embodiments, the lobes 1052 can define a non-circular aperture profile (e.g., a wave-shaped, multi-lobed, or generally polygonal-with-rounded-corners profile) that increases the likelihood of multi-point contact at off-axis orientations, thereby maintaining locking engagement and reducing angular play over a predetermined cone of angulation.

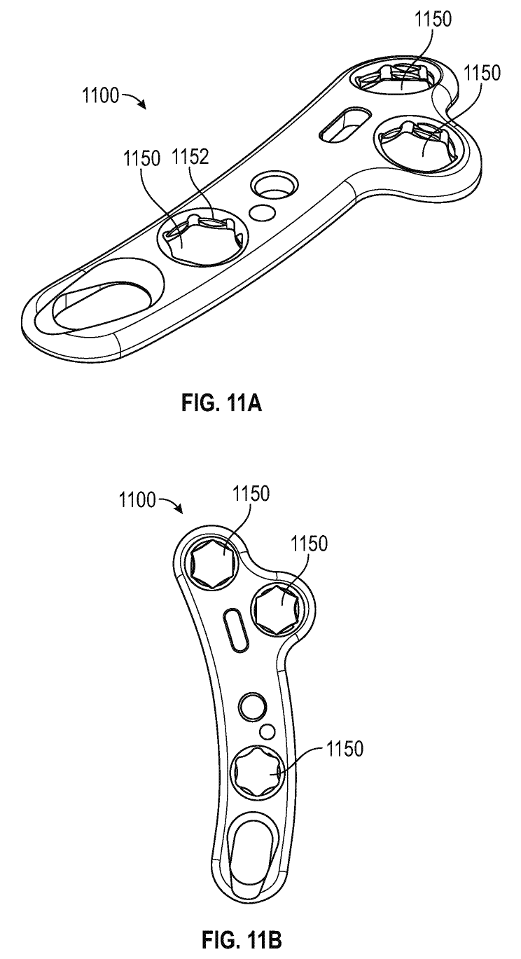

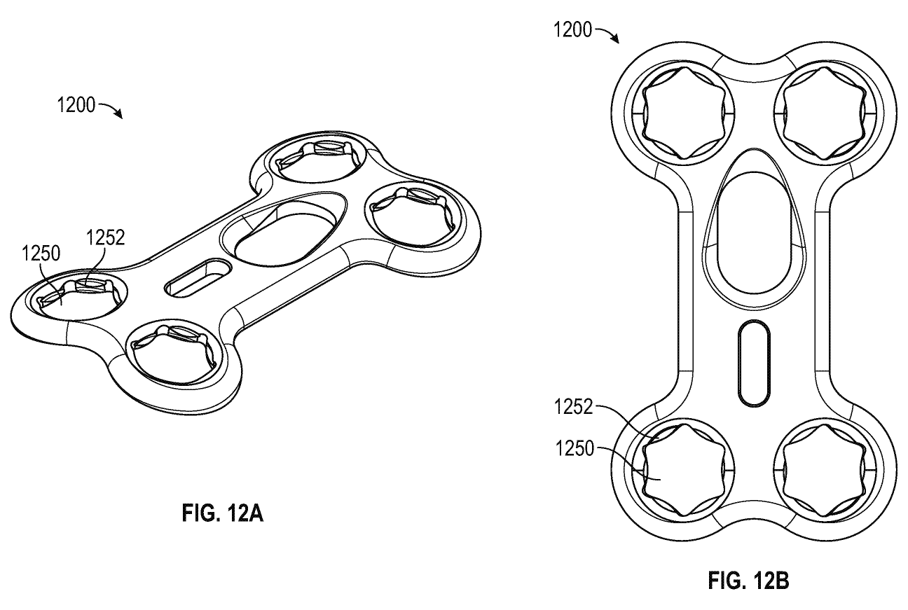

FIG. 11A shows a perspective view of an example of a bone plate 1100. FIG. 11B shows a top view of the example of the bone plate 1100.

The bone plate 1100 can include any of the features of the bone plate 1000 as described with respect to FIGS. 10A-10B.

In some embodiments, the bone plate 1100 can include 3 openings 1150 for securing a bone screw to the bone plate 1100. Each opening 1150 can include lobes 1152 for interacting with the screw head. The bone plate 1100 can be optimized for securing particular bones or portions of a particular bone in place with respect to each other. The bone plate 1100 can be curved to accommodate the shape of the anatomy. The openings 1150 can be positioned to optimize the securement between the particular bones or portions of bone.

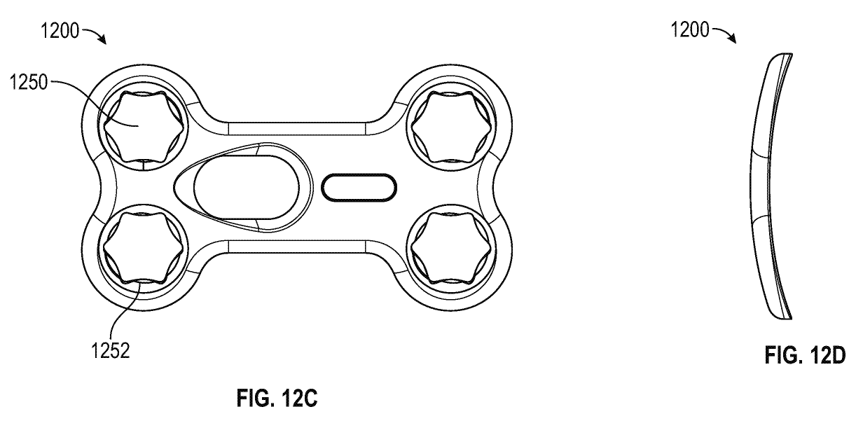

FIG. 12A shows a perspective view of an example of a bone plate 1200. FIG. 12B shows a top view of the example of the bone plate 1200. FIG. 12C shows a top view of the example of the bone plate 1200. FIG. 12D shows a side view of the example of the bone plate 1200.

The bone plate 1200 can include any of the features of the bone plates 1000,1100 as described herein.

In some embodiments, the bone plate 1200 can include 4 openings 1250 for securing a bone screw to the bone plate 1200. Each opening 1250 can include lobes 1252 for interacting with the screw head. The bone plate 1200 can be optimized for securing particular bones or portions of a particular bone in place with respect to each other. In some embodiments, the bone plate 1200 can be curved to accommodate the shape of the anatomy. The openings 1250 can be positioned to optimize the securement between the particular bones or portions of bone.

As shown in FIG. 12D, the bone plate 1200 can be curved to accommodate the anatomy and secure the bone screws at a certain angle. Each opening 1250 can be curved along the plane of the bone plate 1200.

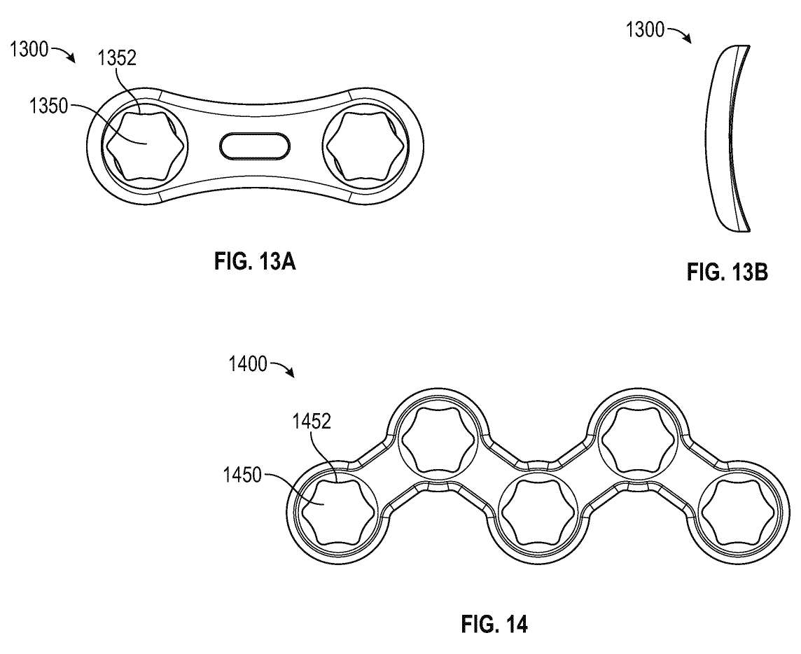

FIG. 13A shows a top view of an example of a bone plate 1300. FIG. 13B a side view of the example of the bone plate 1300.

In some embodiments, the bone plate 1300 can include any of the features of the bone plates 1000, 1100, 1200 as described herein.

In some embodiments, the bone plate 1300 can include 2 openings 1350 for securing a bone screw to the bone plate 1300. In some embodiments, the bone plate 1300 can include 1-10 openings 1350 for securing a bone screw to the bone plate 1300. Each opening 1350 can include lobes 1352 for interacting with the screw head. The bone plate 1300 can be optimized for securing particular bones or portions of a particular bone in place with respect to each other. The bone plate 1300 can be curved to accommodate the shape of the anatomy. The openings 1350 can be positioned to optimize the securement between the particular bones or portions of bone.

As shown in FIG. 13B, the bone plate 1300 can be curved to accommodate the anatomy and secure the bone screws at a certain angle. Each opening 1350 can be curved along the plane of the bone plate 1300.

FIG. 14 shows a top view of an example of a bone plate 1400.

The bone plate 1400 can include any of the features of the bone plates 1000, 1100, 1200, 1300 as described herein.

In some embodiments, the bone plate 1400 can include 5 openings 1450 for securing a bone screw to the bone plate 1400. Each opening 1450 can include lobes 1452 for interacting with the screw head. The bone plate 1400 can be optimized for securing particular bones or portions of a particular bone in place with respect to each other. The bone plate 1400 can be curved to accommodate the shape of the anatomy. The openings 1450 can be positioned to optimize the securement between the particular bones or portions of bone. The bone plate 1400 can have a zigzag arrangement.

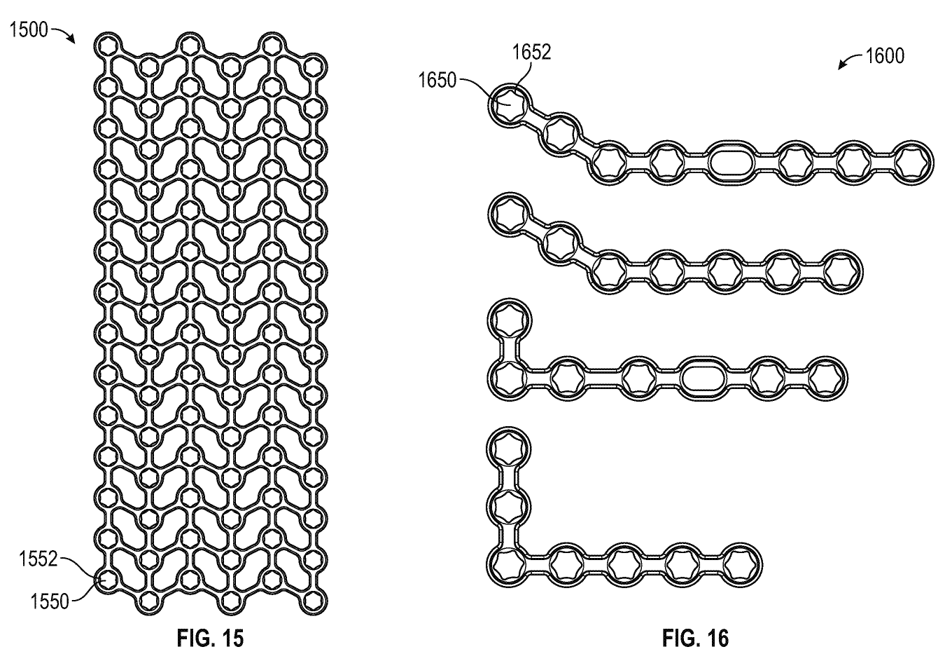

FIG. 15 shows a top view of an example of a bone plate 1500.

The bone plate 1500 can include any of the features of the bone plates 1000, 1100, 1200, 1300, 1400 as described herein.

In some embodiments, the bone plate 1500 can be a mesh plate. In some embodiments, the bone plate 1500 can include 84 openings 1550 for securing a bone screw to the bone plate 1500. In some embodiments, the bone plate 1500 can include 60-100 openings 1550 for securing a bone screw to the bone plate 1500. In some embodiments, the bone plate 1500 can include 40-120 openings 1550 for securing a bone screw to the bone plate 1500. Each opening 1550 can include lobes 1552 for interacting with the screw head. The bone plate 1500 can be optimized for securing particular bones or portions of a particular bone in place with respect to each other. The bone plate 1500 can be curved to accommodate the shape of the anatomy. The openings 1550 can be positioned to optimize the securement between the particular bones or portions of bone. In some embodiments, the bone plate 1500 can be arranged in 6 columns of openings 1550. In some embodiments, the bone plate 1500 can be arranged in 2-10 columns of openings 1550. In some embodiments, each column of openings 1550 can be unaligned with an adjacent column, but aligned with the next non-adjacent column.

FIG. 16 shows a top view of an example of a bone plate 1600.

The bone plate 1600 can include any of the features of the bone plates 1000, 1100, 1200, 1300, 1400, 1500 as described herein.

In some embodiments, the bone plate 1600 can be an L-shaped plate. In some embodiments, the bone plate 1600 can include 5-8 openings 1650 for securing a bone screw to the bone plate 1600. Each opening 1650 can include lobes 1652 for interacting with the screw head. The bone plate 1600 can be optimized for securing particular bones or portions of a particular bone in place with respect to each other. The bone plate 1600 can be curved to accommodate the shape of the anatomy. The openings 1650 can be positioned to optimize the securement between the particular bones or portions of bone. Each segment of the bone plate 1600 can be arranged as an angle shape. Each segment of the bone plate 1600 can be arranged as a right angle or obtuse angle.

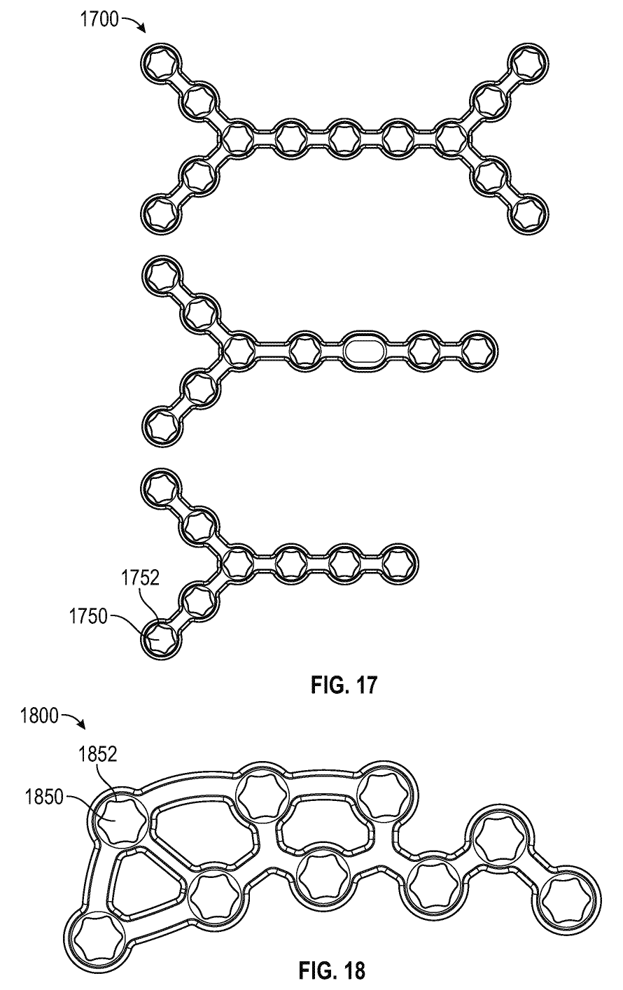

FIG. 17 shows a top view of an example of a bone plate 1700.

The bone plate 1700 can include any of the features of the bone plates 1000, 1100, 1200, 1300, 1400, 1500, 1600 as described herein.

In some embodiments, the bone plate 1700 can be a Y-shaped plate. In some embodiments, the bone plate 1700 can include 10-15 openings 1750 for securing a bone screw to the bone plate 1700. Each opening 1750 can include lobes 1752 for interacting with the screw head. The bone plate 1700 can be optimized for securing particular bones or portions of a particular bone in place with respect to each other. The bone plate 1700 can be curved to accommodate the shape of the anatomy. The openings 1750 can be positioned to optimize the securement between the particular bones or portions of bone. Each segment of the bone plate 1700 can be arranged in the shape of connected straight and diagonal lines.

FIG. 18 shows a top view of an example of a bone plate 1800.

The bone plate 1800 can include any of the features of the bone plates 1000, 1100, 1200, 1300, 1400, 1500, 1600, 1700 as described herein.

In some embodiments, the bone plate 1800 can be a navicular plate. In some embodiments, the bone plate 1800 can include 9 openings 1850 for securing a bone screw to the bone plate 1800. In some embodiments, the bone plate 1800 can include 5-15 openings 1850 for securing a bone screw to the bone plate 1800. Each opening 1850 can include lobes 1852 for interacting with the screw head. The bone plate 1800 can be optimized for securing particular bones or portions of a particular bone in place with respect to each other. The bone plate 1800 can be curved to accommodate the shape of the anatomy. The openings 1850 can be positioned to optimize the securement between the particular bones or portions of bone. The bone plate 1800 can have a shape made up of multiple triangles and angles with openings 1850 at the corners.

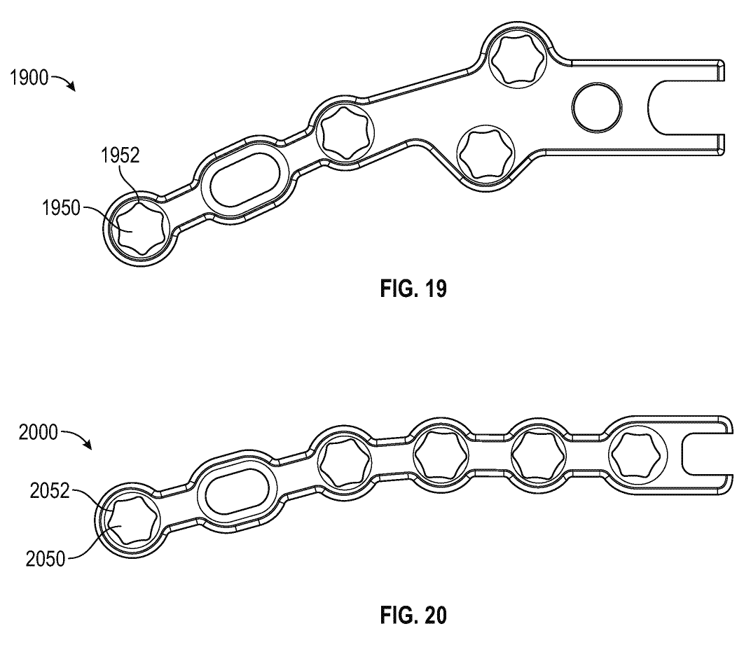

FIG. 19 shows a top view of an example of a bone plate 1900.

The bone plate 1900 can include any of the features of the bone plates 1000, 1100, 1200, 1300, 1400, 1500, 1600, 1700, 1800 as described herein.

In some embodiments, the bone plate 1900 can be a 5^{th} metatarsal plate. In some embodiments, the bone plate 1900 can include 4 openings 1950 for securing a bone screw to the bone plate 1900. Each opening 1950 can include lobes 1952 for interacting with the screw head. The bone plate 1900 can be optimized for securing particular bones or portions of a particular bone in place with respect to each other. The bone plate 1900 can be curved to accommodate the shape of the anatomy. The openings 1950 can be positioned to optimize the securement between the particular bones or portions of bone. The bone plate 1900 can have a curved linear shape.

FIG. 20 shows a top view of an example of a bone plate 2000.

The bone plate 2000 can include any of the features of the bone plates 1000, 1100, 1200, 1300, 1400, 1500, 1600, 1700, 1800, 1900 as described herein.

In some embodiments, the bone plate 2000 can be a 5^{th} metatarsal plate. In some embodiments, the bone plate 2000 can include 5 openings 2050 for securing a bone screw to the bone plate 2000. Each opening 2050 can include lobes 2052 for interacting with the screw head. The bone plate 2000 can be optimized for securing particular bones or portions of a particular bone in place with respect to each other. The bone plate 2000 can be curved to accommodate the shape of the anatomy. The openings 2050 can be positioned to optimize the securement between the particular bones or portions of bone. The bone plate 2000 can have a curved linear shape.

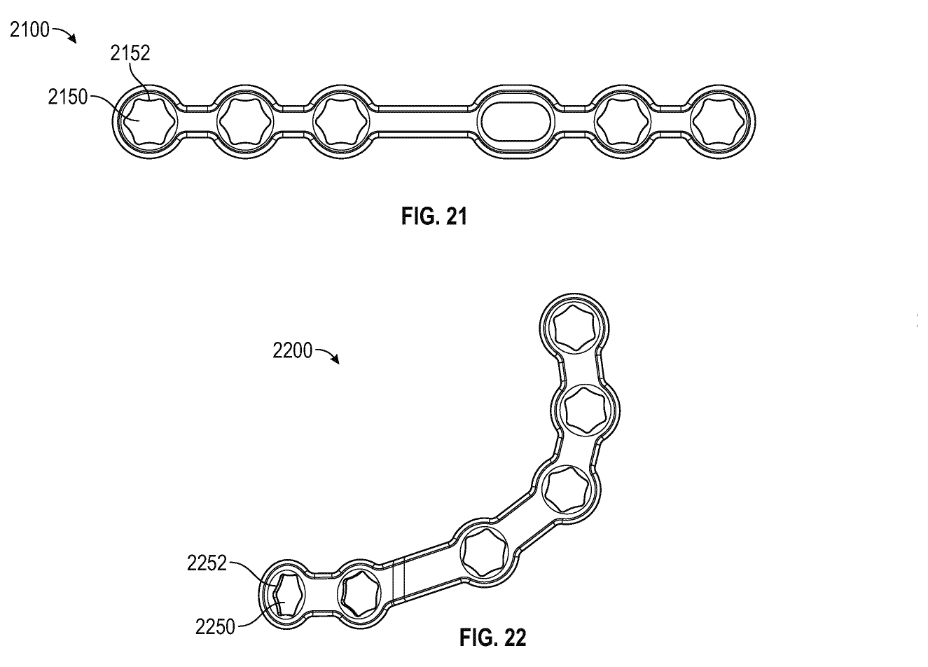

FIG. 21 shows a top view of an example of a bone plate 2100.

The bone plate 2100 can include any of the features of the bone plates 1000,1100,1200,1300,1400,1500,1600,1700,1800,1900,2000 as described herein.

In some embodiments, the bone plate 2100 can include 5 openings 2150 for securing a bone screw to the bone plate 2100. Each opening 2150 can include lobes 2152 for interacting with the screw head. The bone plate 2100 can be optimized for securing particular bones or portions of a particular bone in place with respect to each other. The bone plate 2100 can be curved to accommodate the shape of the anatomy. The openings 2150 can be positioned to optimize the securement between the particular bones or portions of bone. The bone plate 2100 can have a linear shape.

FIG. 22 shows a top view of an example of a bone plate 2200.

The bone plate 2200 can include any of the features of the bone plates 1000,1100,1200,1300,1400,1500,1600,1700,1800,1900,2000,2100 as described herein.

In some embodiments, the bone plate 2200 can be a talar neck plate. In some embodiments, the bone plate 2200 can include 6 openings 2250 for securing a bone screw to the bone plate 2200. Each opening 2250 can include lobes 2252 for interacting with the screw head. The bone plate 2200 can be optimized for securing particular bones or portions of a particular bone in place with respect to each other. The bone plate 2200 can be curved to accommodate the shape of the anatomy. The openings 2250 can be positioned to optimize the securement between the particular bones or portions of bone. The bone plate 2200 can have a curved linear shape.

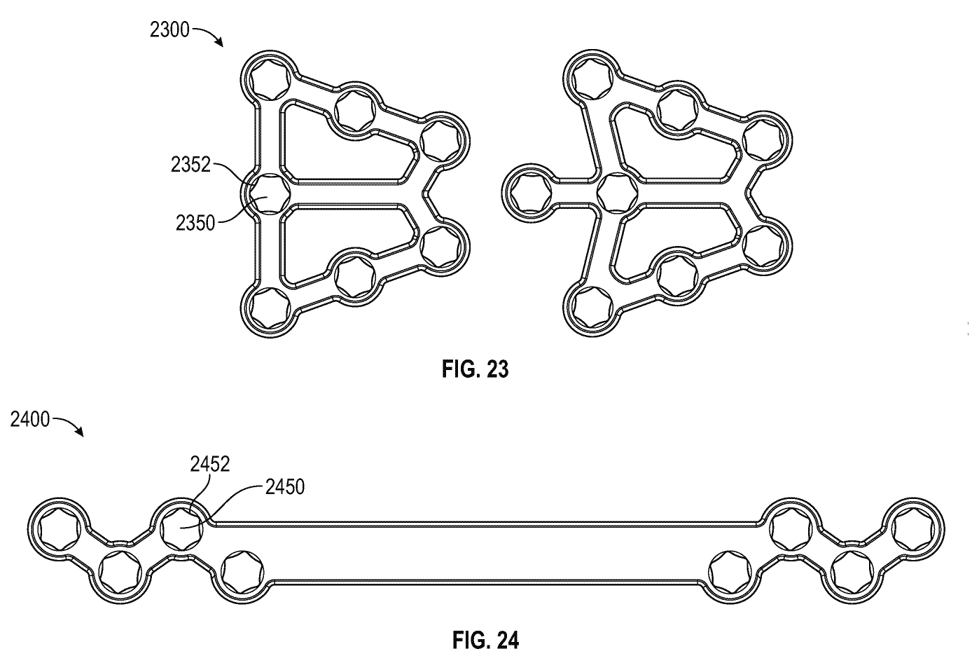

FIG. 23 shows a top view of an example of a bone plate 2300.

The bone plate 2300 can include any of the features of the bone plates 1000, 1100, 1200, 1300, 1400, 1500, 1600, 1700, 1800, 1900, 2000, 2100, 2200 as described herein.

In some embodiments, the bone plate 2300 can include 16 openings 2350 for securing a bone screw to the bone plate 2300. In some embodiments, the bone plate 2300 can include 10-20 openings 2350 for securing a bone screw to the bone plate 2300. The bone plate 2300 can include two separate segments. Each opening 2350 can include lobes 2352 for interacting with the screw head. The bone plate 2300 can be optimized for securing particular bones or portions of a particular bone in place with respect to each other. The bone plate 2300 can be curved to accommodate the shape of the anatomy. The openings 2350 can be positioned to optimize the securement between the particular bones or portions of bone. Each segment of the bone plate 2300 can have a shape formed by straight and diagonal lines.

FIG. 24 shows a top view of an example of a bone plate 2400.

The bone plate 2400 can include any of the features of the bone plates 1000, 1100, 1200, 1300, 1400, 1500, 1600, 1700, 1800, 1900, 2000, 2100, 2200, 2300 as described herein.

In some embodiments, the bone plate 2400 can include 8 openings 2450 for securing a bone screw to the bone plate 2400. In some embodiments, the bone plate 2400 can include 4-12 openings 2450 for securing a bone screw to the bone plate 2400. Each opening 2450 can include lobes 2452 for interacting with the screw head. The bone plate 2400 can be optimized for securing particular bones or portions of a particular bone in place with respect to each other. The bone plate 2400 can be curved to accommodate the shape of the anatomy. The openings 2450 can be positioned to optimize the securement between the particular bones or portions of bone. The bone plate 2400 can have a substantially linear shape.

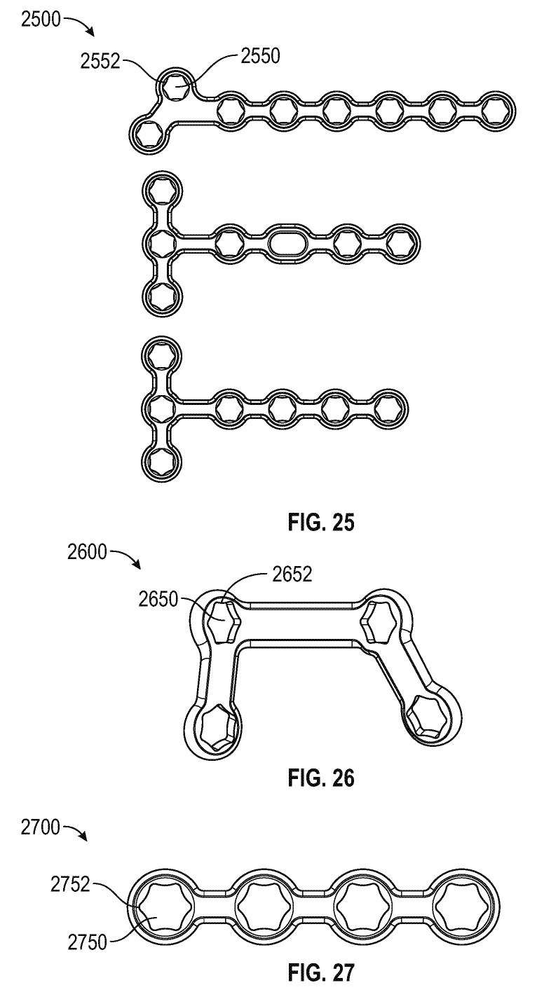

FIG. 25 shows a top view of an example of a bone plate 2500.

The bone plate 2500 can include any of the features of the bone plates 1000, 1100, 1200, 1300, 1400, 1500, 1600, 1700, 1800, 1900, 2000, 2100, 2200, 2300, 2400 as described herein.

In some embodiments, the bone plate 2500 can include 5-10 openings 2550 for securing a bone screw to the bone plate 2500. Each opening 2550 can include lobes 2552 for interacting with the screw head. The bone plate 2500 can be optimized for securing particular bones or portions of a particular bone in place with respect to each other. The bone plate 2500 can be curved to accommodate the shape of the anatomy. The openings 2550 can be positioned to optimize the securement between the particular bones or portions of bone. Each segment of the bone plate 2500 can have be made up of substantially linear shapes.

FIG. 26 shows a top view of an example of a bone plate 2600.

The bone plate 2600 can include any of the features of the bone plates 1000, 1100, 1200, 1300, 1400, 1500, 1600, 1700, 1800, 1900, 2000, 2100, 2200, 2300, 2400, 2500 as described herein.

In some embodiments, the bone plate 2600 can be a talar neck plate. In some embodiments, the bone plate 2600 can include 4 openings 2650 for securing a bone screw to the bone plate 2600. Each opening 2650 can include lobes 2652 for interacting with the screw head. The bone plate 2600 can be optimized for securing particular bones or portions of a particular bone in place with respect to each other. The bone plate 2600 can be curved to accommodate the shape of the anatomy. The openings 2650 can be positioned to optimize the securement between the particular bones or portions of bone. The bone plate 2600 can be formed of two substantially linear shapes connected by a surface that is not parallel or perpendicular to the linear shapes.

FIG. 27 shows a top view of an example of a bone plate 2700.

The bone plate 2700 can include any of the features of the bone plates 1000, 1100, 1200, 1300, 1400, 1500, 1600, 1700, 1800, 1900, 2000, 2100, 2200, 2300, 2400, 2500, 2600 as described herein.

In some embodiments, the bone plate 2700 can include 4 openings 2750 for securing a bone screw to the bone plate 2700. Each opening 2750 can include lobes 2752 for interacting with the screw head. The bone plate 2700 can be optimized for securing particular bones or portions of a particular bone in place with respect to each other. The bone plate 2700 can be curved to accommodate the shape of the anatomy. The openings 2750 can be positioned to optimize the securement between the particular bones or portions of bone. The bone plate 2700 can be substantially linear.

### Interaction of Bone Screws and Plates:

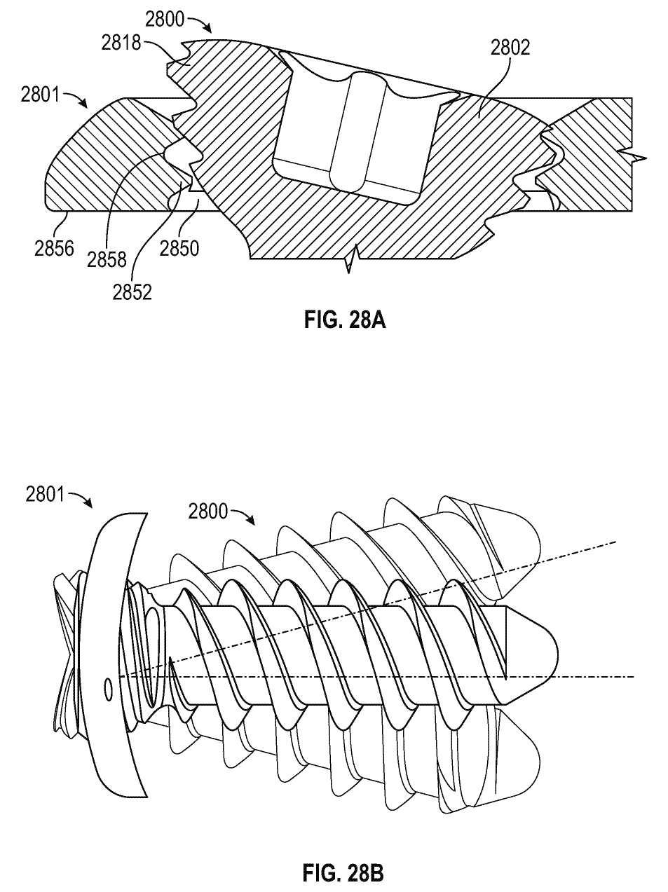

FIG. 28A shows an example of a head 2802 of a bone screw 2800 engaged with a bone plate 2801. FIG. 28B shows an example of the bone screw 2800 engaged with the bone plate 2801 to illustrate the cone of angulation allowed by the interaction.

The bone screw 2800 can include any of the features of the bone screw 100, 200, 300, 400, 500, 600, 700, 800, 900 as described herein. The bone plate 2801 can include any of the features of the bone plate 1000, 1100, 1200, 1300, 1400, 1500, 1600, 1700, 1800, 1900, 2000, 2100, 2200, 2300, 2400, 2500, 2600, 2700 as described herein.

The bone screw 2800 can have a head 2802 with threads 2818. The bone plate 2801 can have lobes 2852 in the opening 2850 with threads 2858.

In some embodiments, the head 2802 of the bone screw 2800 can have a thread 2818 with a pitch of 20° or approximately 20°. In some embodiments, the head 2802 of the bone screw 2800 can have a thread 2818 with a pitch of from approximately 10° to approximately 30°. In some embodiments, the head 2802 of the bone screw 2800 can have a thread 2818 with a pitch of from approximately 5° to approximately 45°. In some embodiments, the lobes 2852 of the bone plate 2801 can have a thread 2858 with a pitch of approximately 5°. In some embodiments, the lobes 2852 of the bone plate 2801 can have a thread 2858 with a pitch of from approximately 1° to approximately 10°. In some embodiments, the lobes 2852 of the bone plate 2801 can have a thread 2858 with a pitch of from approximately 1° approximately 20°. Advantageously, for example, a screw head 2802 with a thread 2818 of 20 degrees and a bone plate 2801 with a thread 2858 of 5° can allow for a 30° cone of angulation. In some embodiments, the cone of angulation can be at least 20° and/or less than or equal to 40°. In some embodiments, the cone of angulation can be at least 15° and/or less than or equal to 45°. In some embodiments, the cone of angulation can be at least 5° and/or less than or equal to 55°. In some embodiments, the cone of angulation can be at least 0° and/or less than or equal to 60°.

Advantageously, in some embodiments, the interaction of the screw head 2802 with the bone plate 2801 can ensure high pull-out strength, low movement (or wobbling) of the screw, and high strength with a minimized system profile. Minimized profile can mean the total height of the plate and screw system from the bottom surface 2856 of the bone plate 2801 to the highest point of the screw head 2802. In some embodiments, the opening 2850 in the bone plate 2801 may plastically deform as the bone screw 2800 is inserted. In some embodiments, the bone screw 2800 can engage the bone plate 2801 for 2 full threads. In some embodiments, the bone screw 2800 can engage the bone plate 2801 for 1-3 full threads. In some embodiments, the bone screw 2800 can engage the bone plate 2801 for 1-5 full threads. In some embodiments, the bone screw 2800 can engage the bone plate 2801 for 1-10 full threads.

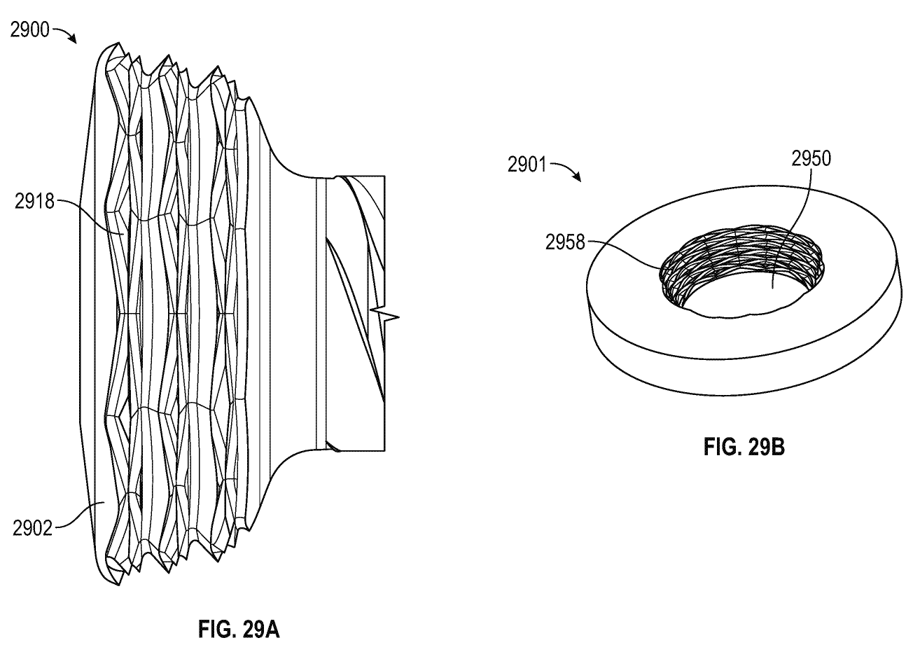

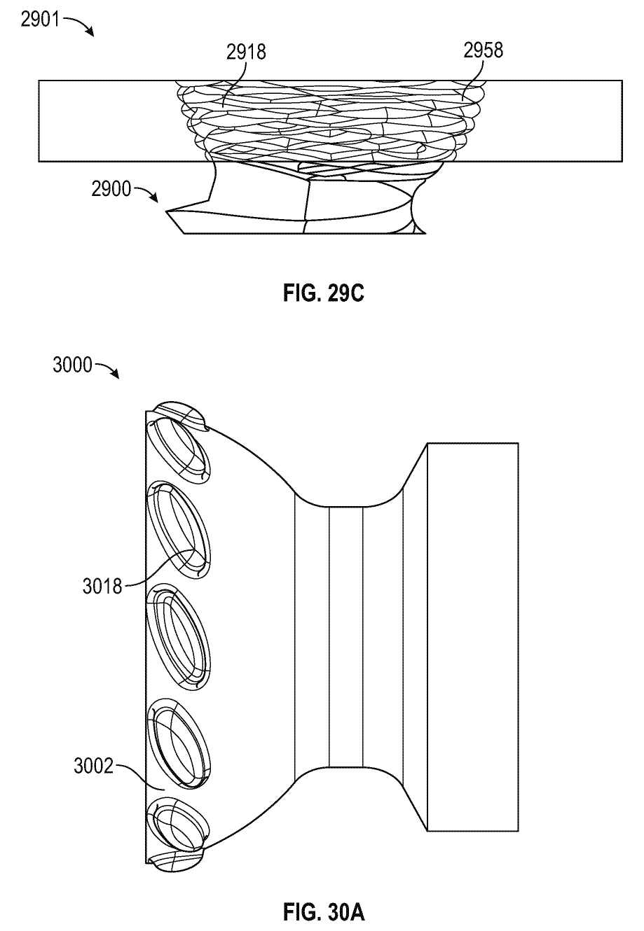

FIG. 29A shows an example of a head 2902 of a bone screw 2900 with diamond-shaped threads 2918. FIG. 29B shows an example of a bone plate 2901 with diamond-shaped threads 2958. FIG. 29C shows an example of the head 2902 of the bone screw 2900 interacting with the bone plate 2901 with diamond-shaped threads 2958.

The bone screw 2900 can include any of the features of the bone screw 100, 200, 300, 400, 500, 600, 700, 800, 900, 2800 as described herein. The bone plate 2801 can include any of the features of the bone plate 1000, 1100, 1200, 1300, 1400, 1500, 1600, 1700, 1800, 1900, 2000, 2100, 2200, 2300, 2400, 2500, 2600, 2700, 2801 as described herein.

In some embodiments, the diamond-shaped threads 2918 of the screw head 2902 can interact with the corresponding diamond-shaped threads 2958 of the bone plate 2901 to improve securement of the bone screw 2900 to the bone plate 2901. In some embodiments, the diamond-shaped threads 2918, 2958 can be formed as elongated diamond shapes around the perimeter of the bone screw 2900 and within the perimeter of the opening 2950 of the bone plate 2901, respectively. The diamond-shaped threads 2918, 2958 can allow for a variety of angles of positioning the bone screw 2900 in the bone plate 2901.

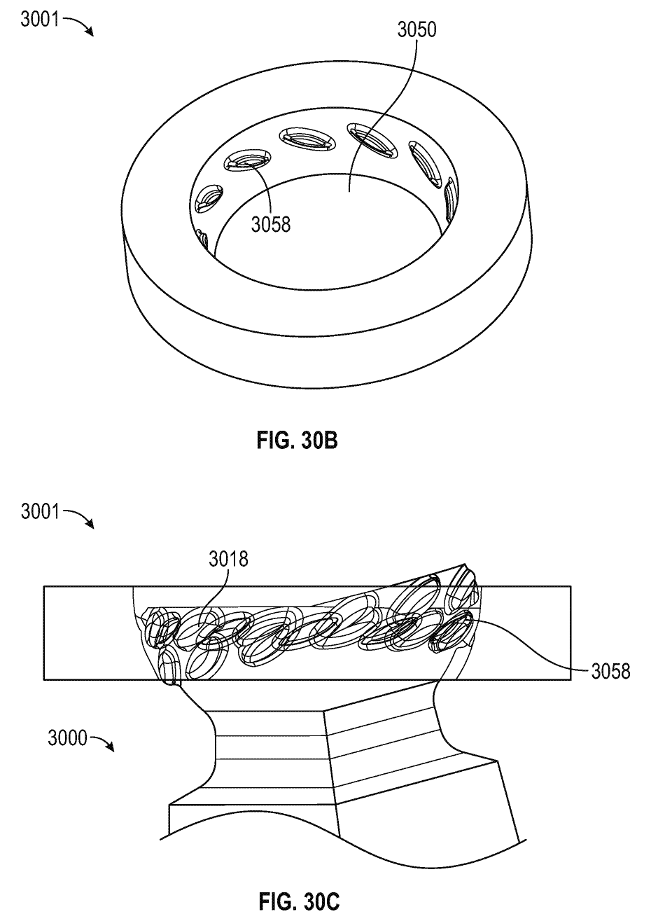

FIG. 30A shows an example of a head 3002 of a bone screw 3000 with elliptical engagement features 3018. FIG. 30B shows an example of a bone plate 3001 with elliptical engagement features. FIG. 30C shows an example of the head 3002 of the bone screw 3000 interacting with the bone plate 3001 with elliptical engagement features 3058.

The bone screw 3000 can include any of the features of the bone screw 100, 200, 300, 400, 500, 600, 700, 800, 900, 2800, 2900 as described herein. The bone plate 2801 can include any of the features of the bone plate 1000, 1100, 1200, 1300, 1400, 1500, 1600, 1700, 1800, 1900, 2000, 2100, 2200, 2300, 2400, 2500, 2600, 2700, 2801, 2901 as described herein.

In some embodiments, the elliptical engagement features 3018 of the screw head 3002 can interact with the corresponding elliptical engagement features 3058 of the bone plate 3001 to improve securement of the bone screw 3000 to the bone plate 3001. In some embodiments, the elliptical engagement features 3018,3058 can be formed as diagonal ellipses around the perimeter of the bone screw 3000 and within the perimeter of the opening 3050 of the bone plate 3001, respectively. The elliptical engagement features 3018,3058 can allow for a variety of angles of positioning the bone screw 3000 in the bone plate 3001.

### Calcaneus Bone Plate:

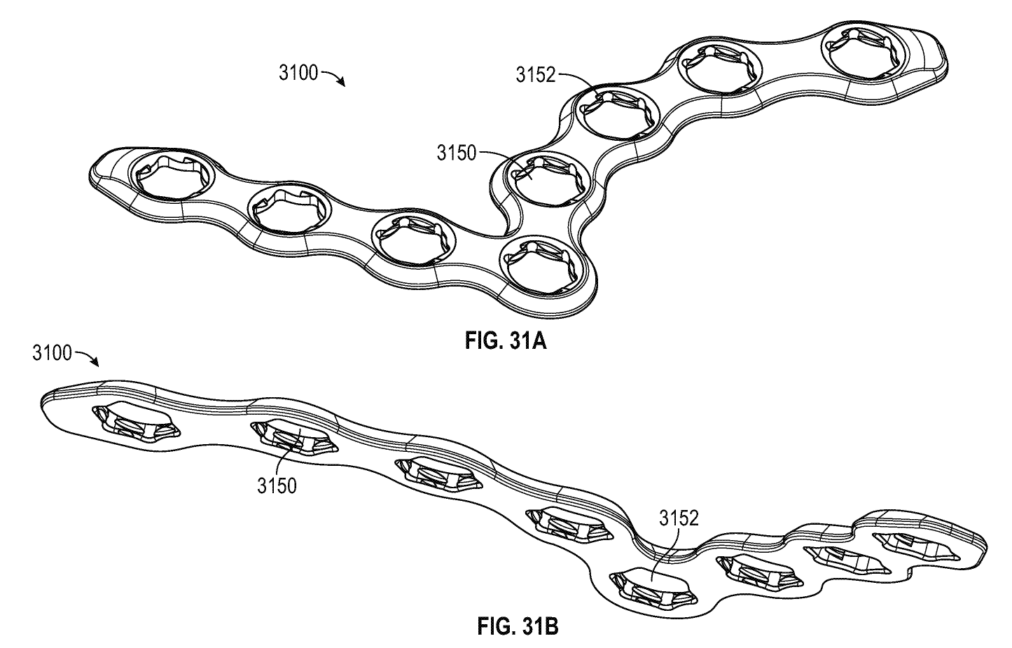

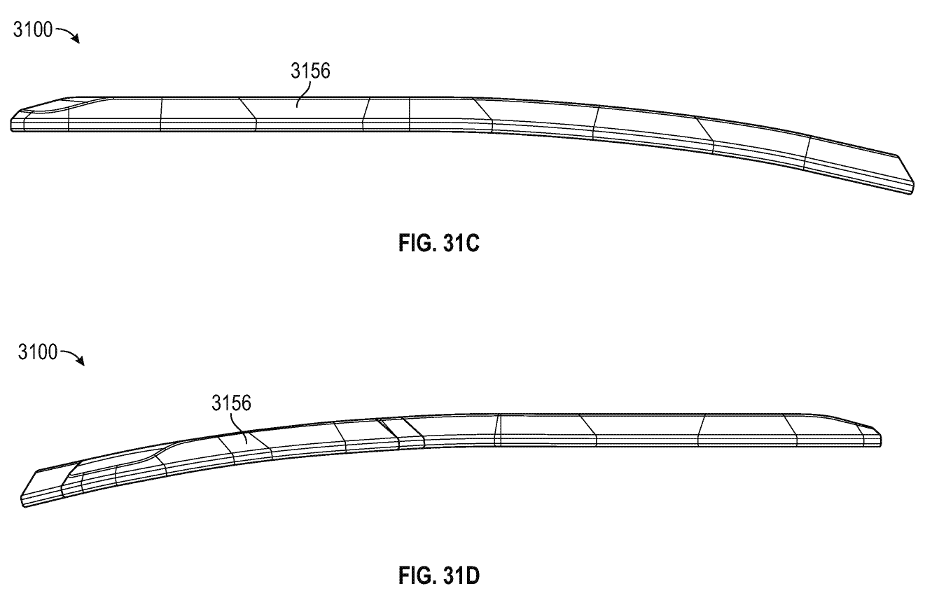

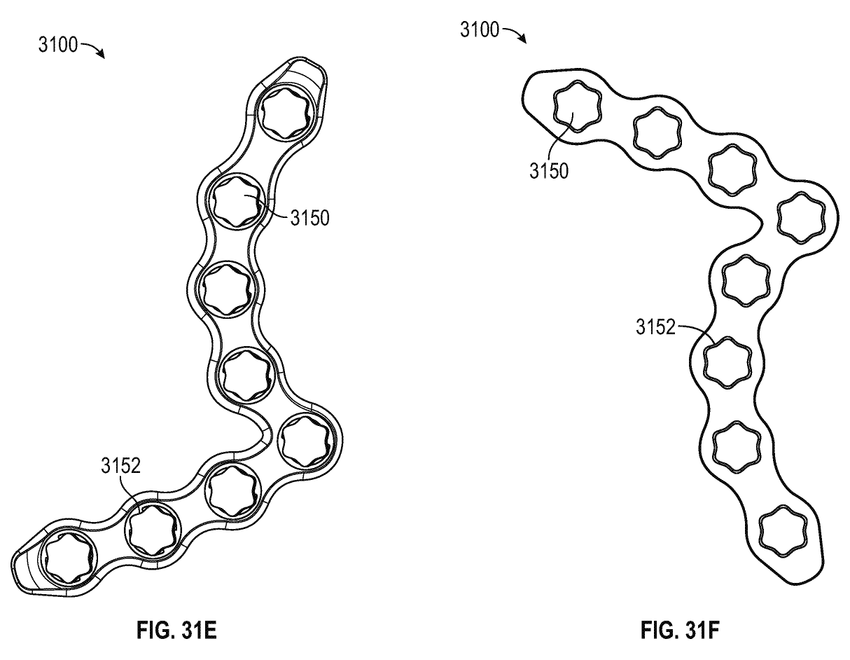

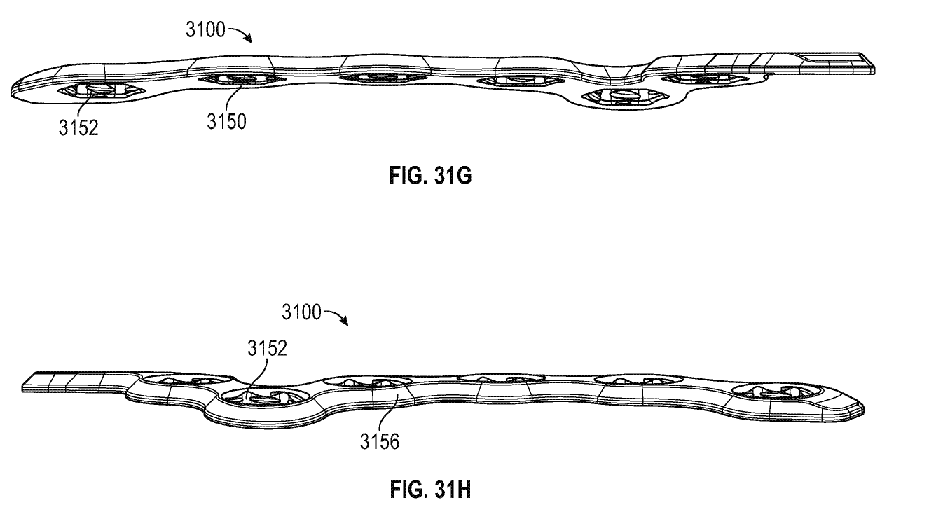

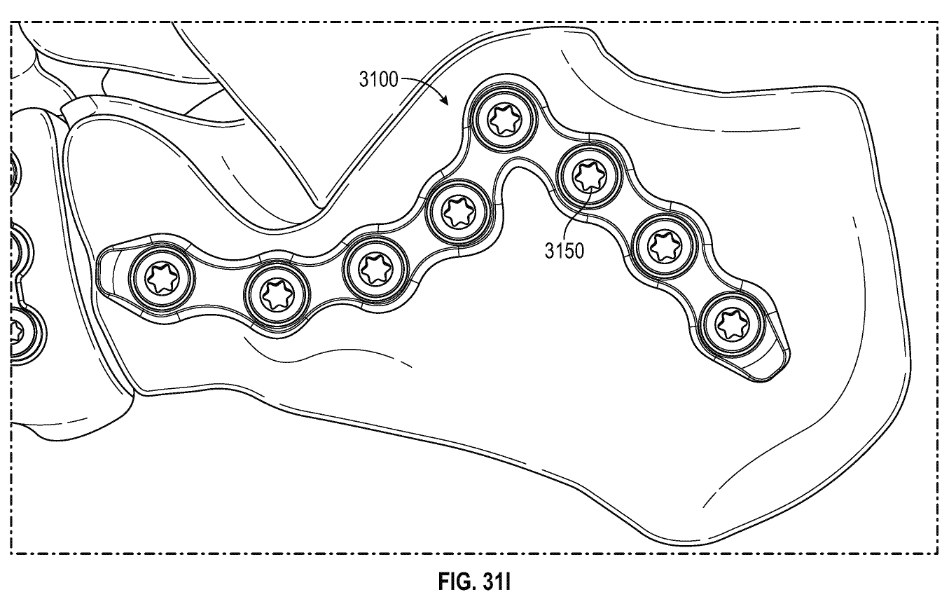

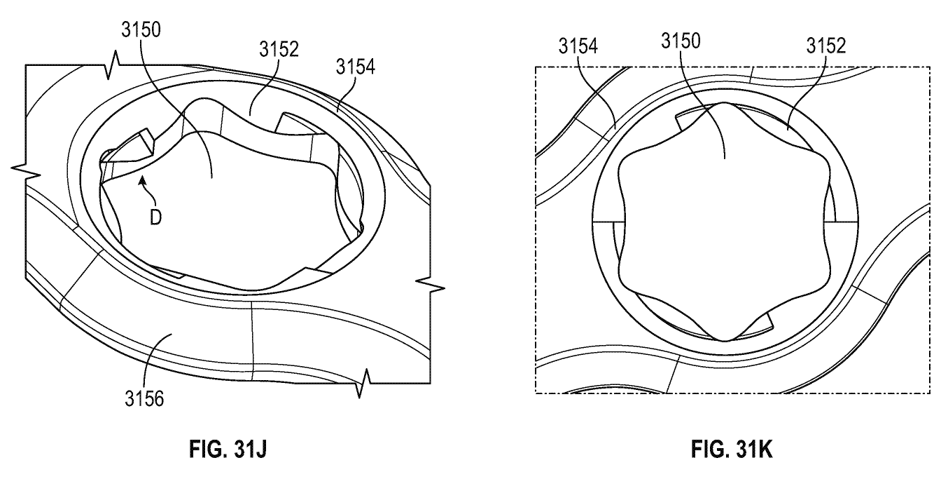

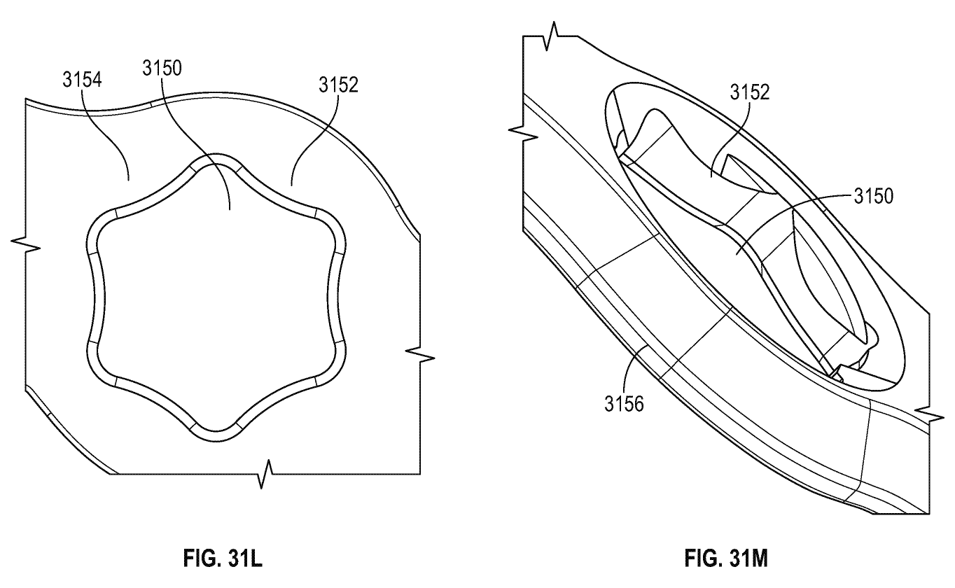

FIG. 31A shows a top perspective view an example of a bone plate 3100. FIG. 31B shows a bottom perspective view of the example of the bone plate 3100 of FIG. 31A. FIGS. 31C-31D show side views of the example of the bone plate 3100 of FIG. 31A. FIG. 31E shows a top view of the example of the bone plate of FIG. 31A. FIG. 31F shows a bottom view of the example of the bone plate 3100 of FIG. 31A. FIG. 31G shows a back view of the example of the bone plate 3100 of FIG. 31A. FIG. 31H shows a front view of the example of the bone plate 3100 of FIG. 31A. FIG. 31I shows the example of the bone plate 3100 of FIG. 31A on a calcaneus. FIG. 31J shows a perspective view of the opening 3150 of the bone plate 3100 of FIG. 31A. FIG. 31K shows a top view of the opening 3150 of the bone plate 3100 of FIG. 31A. FIG. 31L shows a bottom view of the opening 3150 of the bone plate 3100 of FIG. 31A. FIG. 31M shows a side perspective view of the opening 3150 of the bone plate 3100 of FIG. 31A.

The bone plate 3100 can include any of the features of the bone plates 1000, 1100, 1200, 1300, 1400, 1500, 1600, 1700, 1800, 1900, 2000, 2100, 2200, 2300, 2400, 2500, 2600, 2700 as described herein.

In some embodiments, the bone plate 3100 may be used for fixation of a calcaneus or heel bone. In some embodiments, the bone plate 3100 can include 8 openings 3150 for securing a bone screw to the bone plate 3100. In some embodiments, the bone plate 3100 can include 5-10 openings 3150 for securing a bone screw to the bone plate 3100. Each opening 3150 can include lobes 3152 for interacting with the screw head. The bone plate 3100 can be curved to accommodate the shape of the anatomy. The openings 3150 can be positioned to optimize the securement between the particular bones or portions of bone, for example of the calcaneus. The bone plate 3100 can have a linear segment and a curved or bended segment.

### Other Embodiments:

Disclosed below are non-limiting examples of the systems, methods, and devices described herein.

Example 1. A bone screw configured to be secured to a bone comprising: a shaft having a proximal end and a distal end, wherein at least the distal end of the shaft has a trilobe shaped profile; a thread on the shaft, the thread comprising: a first thread portion having a first leading surface disposed at a first thread angle with respect to the shaft; a second thread portion having a second leading surface disposed at a second thread angle with respect to the shaft, wherein the second thread angle is greater than the first thread angle, and wherein the second thread portion is proximal to the first thread portion; and a transition portion between the first thread portion and the second thread portion, the transition portion comprising a taper in the thread.

Example 2. The bone screw of Example 1, wherein the transition portion comprises an undercut on the thread.

Example 3. The bone screw of any one of Examples 1 or 2, further comprising a cutting flute on the distal end of the shaft.

Example 4. The bone screw of any one of Examples 1-3, wherein the second thread portion has a greater volume per surface area than the first thread portion.

Example 5. The bone screw of any one of Examples 1-4, wherein the second thread portion has a greater volume per surface area than the first thread portion.

Example 6. The bone screw of any one of Examples 1-5, further comprising three sharp points on a distalmost portion of the distal end.

Example 7. The bone screw of any one of Examples 1-6, wherein the second thread portion has a smaller crest width than the first thread portion.

Example 8. The bone screw of any one of Examples 1-7, wherein the thread has a wavy shape.

Example 9. The bone screw of any one of Examples 1-8, wherein the thread comprises serrated lips.

Example 10. The bone screw of any one of Examples 1-9, further comprising a lumen extending longitudinally through a center of the shaft from the proximal end to the distal end.

Example 11. A bone plate configured to support a bone comprising: a main body; an opening in the main body, wherein at least an upper portion and a lower portion of the opening comprise a substantially circular shape; a plurality of lobes extending from an inner surface of the opening; and a concave surface between each lobe of the plurality of lobes, wherein the plurality of lobes are threaded.

Example 12. The bone plate of Example 11, wherein the plurality of lobes comprise two thread starts.

Example 13. The bone plate of Example 12, wherein a first thread start of the two thread starts is on a first lobe of the plurality of lobes and a second thread start of the two thread starts is on a second lobe of the plurality of lobes.

Example 14. The bone plate of Example 13, wherein the first lobe is opposite the second lobe along a diameter of the opening.

Example 15. The bone plate of any one of Examples 11-14, wherein the plurality of lobes comprises six lobes.

Example 16. The bone plate of any one of Examples 11-15, wherein the plurality of lobes are beneath a top surface of the main body and above a bottom surface of the main body.

Example 17. The bone plate of any one of Examples 11-16, further comprising a second opening with a second plurality of lobes.

Example 18. A system for securing bone comprising: a bone screw comprising a trilobe shaped tip; and a bone plate of any one of Examples 11-17, wherein a screw head of the bone screw is configured to engage a plurality of lobes of the bone plate.

Example 19. The system of Example 18, wherein the bone screw is allowed to be positioned in the bone plate at a desired angle between 0° and 15°.

Example 20. A method for securing bone, the method comprising: positioning a bone screw comprising a trilobe shaped tip into a bone plate of any of Examples 11-17, wherein a screw head of the bone screw engages a plurality of lobes of the bone plate.

Example 21. A bone screw configured to be secured to a bone comprising: a shaft; and a thread on the shaft, the thread comprising a thread crest and a thread root; wherein: the thread root is on a first path, the first path being a constant helical path; the thread crest is on a second path; and the second path alternates between a minimum distance and a maximum distance from the first path.

Example 22. A bone screw configured to be secured to a bone comprising: a shaft; and a thread on the shaft, the thread comprising a thread crest and a thread root, wherein the thread root and the thread crest are on a thread path, the thread path alternating between a minimum distance and a maximum distance from a helical path along the shaft.

Example 23. A bone screw configured to be secured to a bone comprising: a shaft; and a thread on the shaft, the thread comprising a series of helical cuts, wherein the helical cuts comprise threaded serrations, the threaded serrations disposed in an axial pattern.

Example 24. A bone plate configured to support a bone, comprising: a main body; an opening in the main body comprising a substantially circular inner surface; a plurality of lobes extending from the inner surface of the opening, the plurality of lobes configured to engage at least one lobe of a screw head of a bone screw; and a concave surface between each lobe of the plurality of lobes, wherein the plurality of lobes are threaded.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above.

Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims.

Any methods disclosed herein need not be performed in the order recited. The methods disclosed herein include certain actions taken by a practitioner; however, they can also include any third-party instruction of those actions, either expressly or by implication. For example, actions such as “applying pressure on a plunger to create an audible and/or tactile click feedback” includes “instructing the applying of pressure on a plunger to create an audible and/or tactile click feedback.”

While certain arrangements of the inventions have been described, these arrangements have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, arrangement, or example are to be understood to be applicable to any other aspect, arrangement or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing arrangements. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some arrangements, the actual steps taken in the processes illustrated and/or disclosed may differ from those shown in the figures. Depending on the arrangement, certain of the steps described above may be removed, others may be added. Furthermore, the features and attributes of the specific arrangements disclosed above may be combined in different ways to form additional arrangements, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular arrangement. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain arrangements include, while other arrangements do not include, certain features, elements, and/or steps. Thus, such conditional language is not generally intended to imply that features, elements, and/or steps are in any way required for one or more arrangements or that one or more arrangements necessarily include logic for deciding, with or without user input or prompting, whether these features, elements, and/or steps are included or are to be performed in any particular arrangement.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain arrangements require the presence of at least one of X, at least one of Y, and at least one of Z.

Language of degree used herein, such as the terms “approximately,” “about,” “generally,” and “substantially” as used herein represent a value, amount, or characteristic equal to or close to the stated value, amount, or characteristic that still performs a desired function or achieves a desired result. For example, the terms “approximately”, “about”, “generally,” and “substantially” may refer to an amount that is within 10% of the stated amount. As another example, in certain arrangements, the terms “generally parallel” and “substantially parallel” refer to a value, amount, or characteristic that departs from exactly parallel by less than or equal to 5°. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred arrangements in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A bone plate configured to support a bone comprising:
a main body;
an opening in the main body, wherein at least an upper portion and a lower portion of the opening comprise a substantially circular shape;
a plurality of lobes extending from an inner surface of the opening; and
a concave surface between each lobe of the plurality of lobes,
wherein the plurality of lobes are threaded.

2. The bone plate of claim 1, wherein the plurality of lobes comprise two thread starts.

3. The bone plate of claim 2, wherein a first thread start of the two thread starts is on a first lobe of the plurality of lobes and a second thread start of the two thread starts is on a second lobe of the plurality of lobes.

4. The bone plate of claim 3, wherein the first lobe is opposite the second lobe along a diameter of the opening.

5. The bone plate of claim 1, wherein the plurality of lobes comprises six lobes.

6. The bone plate of claim 1, wherein the plurality of lobes are beneath a top surface of the main body and above a bottom surface of the main body.

7. The bone plate of claim 1, further comprising a second opening with a second plurality of lobes.

8. The bone plate of claim 7, wherein the opening is in a first segment of the main body and the main body comprises a second segment having the second opening.

9. The bone plate of claim 8, wherein the second segment is disposed at an obtuse angle with respect to the first segment.

10. The bone plate of claim 1, wherein the plurality of lobes comprises between 1 and 6 lobes.

11. A system comprising:
the bone plate of claim 1; and
a bone screw configured to be positioned through the opening of the main body of the bone plate.

12. The system of claim 11, wherein a thread or shaft of the bone screw is configured to engage the plurality of lobes of the bone plate.

13. A bone plate configured to support a bone comprising:
a main body;
an opening in the main body;
a plurality of lobes extending from an inner surface of the opening; and
a concave surface between each lobe of the plurality of lobes,
wherein the plurality of lobes are threaded and the plurality of lobes comprise two thread starts.

14. The bone plate of claim 13, wherein the plurality of lobes comprises between 1 and 6 lobes.

15. The bone plate of claim 13, wherein a first thread start of the two thread starts is on a first lobe of the plurality of lobes and a second thread start of the two thread starts is on a second lobe of the plurality of lobes.

16. The bone plate of claim 15, wherein the first lobe is opposite the second lobe along a diameter of the opening.

17. The bone plate of claim 13, wherein the plurality of lobes comprises six lobes.

18. The bone plate of claim 13, wherein the plurality of lobes are beneath a top surface of the main body and above a bottom surface of the main body.

19. The bone plate of claim 13, further comprising a second opening with a second plurality of lobes.

20. The bone plate of claim 19, wherein the opening is in a first segment of the main body and the main body comprises a second segment having the second opening.
